SEQUENCE LISTING (18 sequences)

SEQ ID NO: 1 - DNA, 24 bp, Artificial Sequence
  Description of Artificial Sequence Synthetic oligonucleotide
ctcgagccatctgctggagacatg

SEQ ID NO: 2 - DNA, 18 bp, Artificial Sequence
  Description of Artificial Sequence Synthetic oligonucleotide
tcaggatctggttcagga

SEQ ID NO: 3 - DNA, 24 bp, Artificial Sequence
  Description of Artificial Sequence Synthetic oligonucleotide
tcaggatctggttcaggatccatg

SEQ ID NO: 4 - protein, 354 aa, Homo sapiens
MSRLLHAEEWAEVKELGDHHRQPQPHHLPQPPPPPQPPATLQAREHPVYPPELSLLDSTDPRAWLAPTLQGICTARAAQYLLHSPELGASEAAAPRDEVDGRGELVRRSSGGASSSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSGGEQPPPPPASCKSDHHHLRTAASYEGGAGNATAAGAQQASGGSQRPTPPGSCRTRFSAPASAGGYSVQLDALHFSTFEDSALTAMMAQKNLSPSLPGSILQPVQEENSKTSPRSHRSDGEFSPHSHYSDSDEAS

SEQ ID NO: 5 - protein, 595 aa, Homo sapiens [sequence omitted]

SEQ ID NO: 6 - protein, 495 aa, Homo sapiens
MDIKNSPSSLNSPSSYNCSQSILPLEHGSIYIPSSYVDSHHEYPAMTFYSPAVMNYSIPSNVTNLEGGPGRQTTSPNVLWPTPGHLSPLVVHRQLSHLYAEPQKSPWCEARSLEHTLPVNRETLKRKVSGNRCASPVTGPGSKRDAHFCAVCSDYASGYHYGVWSCEGCKAFFKRSIQGHNDYICPATNQCTIDKNRRKSCQACRLRKCYEVGMVKCGSRRERCGYRLVRRQRSADEQLHCAGKAKRSGGHAPRVRELLLDALSPEQLVLTLLEAEPPHVLISRPSAPFTEASMMMSLTKLADKELVHMISWAKKIPGFVELSLFDQVRLLESCWMEVLMMGLMWRSIDHPGKLIFAPDLVLDRDEGKCVEGILEIFDMLLATTSRFRELKLQHKEYLCVKAMILLNSSMYPLVTATQDADSSRKLAHLLNAVTDALVWVIAKSGISSQQQSMRLANLLMLLSHVRHARAEKASQTLTSFGMKMETLLPEATMEQ

SEQ ID NO: 7 - DNA, 2221 bp, Homo sapiens
ttaattaaactagtcttaagaagcttgaattccaccatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgatctcgagccatctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagcttgatgaagatctgagctccctggcggaattcggatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgacattaatgctagcggcgcgcc

SEQ ID NO: 8 - DNA, 1096 bp, Artificial Sequence
  Description of Artificial Sequence Synthetic polynucleotide
atactgaccatcttggggacagaatgatctagtttgtgaggaccataattgctttaaactctcagcgtgttttctcatgtcctgaaaatggtgacaaaaatattcgattttcaaggcgactgttgatgattcatttaccaatctaaagcactagtgccccgcactcgtaatccataacttgagatgaacacaccaaattataaatatcttattcacctaatccgtggaaagtttgtaacaccaaaatattaacttctgttcttgatcactcagcctccatagttattcatgttgcatgatctgattcgctcttttagaatttctcatagcgtcaggtactagctcaaaataacaggagcaactgcttctttccatatcccacattgaggaaaacacattttgcaagccgtcttaaagaaacaggtcaacttagtttagggatgaaacttaaaagtaagcttcgtcacaaaaaacacagttcaacaccacctcgcccggctgcaggcagctcagaggacaggaagtgaggccaagacatatttcacgcagcacagactaaaccccagcttttatctttcccgctggtccttaagggaccaaccgtactgattttccgtcatgttccgcgccacttccgttcgcgcactttaattacgtcaggcgtcctctctcgcggtatcatccggttgctgaggccctgtaataaaggtctcgcgaaatttgttctagaggtccaagtttgcttcttagcttactccaccccacccccaacctgtccctccttttctttccaagtcacaaaattctcccctcccctaccccggagtttacggccctcctcctgtttccgatttcagcccggaaccggaagtgtagtgggcggggcccgtcggcggaaaacgcagcggagccagagccggacacggctgtggccgctgcctctacccccgccacggatcgccgggtagtaggactgcgcggctccaggctgagggtcggtccggaggcgggtgggcgcgggtctcacccggattgtccgggtggcaccgttcccggccccaccgggcgccgcgagggatcatgtctacagcctctg

SEQ ID NO: 9 - protein, 8 aa, Artificial Sequence
  Description of Artificial Sequence Synthetic peptide
DYKDDDDK

SEQ ID NO: 10 - DNA, 24 bp, Artificial Sequence
  Description of Artificial Sequence Synthetic oligonucleotide
gattacaaggatgacgatgacaag

SEQ ID NO: 11 - DNA, 20 bp, Artificial Sequence
  Description of Artificial Sequence Synthetic primer
ttgtgtgcctcaaatccatc

SEQ ID NO: 12 - DNA, 22 bp, Artificial Sequence
  Description of Artificial Sequence Synthetic primer
ccttacaaacctactacatacc

SEQ ID NO: 13 - DNA, 20 bp, Artificial Sequence
  Description of Artificial Sequence Synthetic primer
cccgtaatgcagaagaagac

SEQ ID NO: 14 - DNA, 20 bp, Artificial Sequence
  Description of Artificial Sequence Synthetic primer
ggtcagtgccttgttggatg

SEQ ID NO: 15 - DNA, 8174 bp, Artificial Sequence
  Description of Artificial Sequence Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattcgccaccatggactacaaagacgatgatgataaagattacaaagatgacgatgacaaggggtcccgcctgctgcatgcagaagagtgggctgaggtaaaagagttgggggaccaccatcgccatccccagccgcaccacgtcccgccgctgacgccacagccacctgctaccctgcaggcgagagaccttcccgtctacccggcagaactgtccctcctggatagcaccgacccacgcgcctggctgactcccactttgcagggcctctgcacggcacgcgccgcccagtatctgctgcattctcccgagctgggtgcctccgaggccgcggcgccccgggacgaggctgacagccagggtgagctggtaaggagaagcggctgtggcggcctcagcaagagccccgggcccgtcaaagtacgggaacagctgtgcaagctgaagggtggggttgtagtggacgagcttggctgcagccgccagcgagccccttccagcaaacaggtgaatggggtacagaagcaaaggaggctggcagcaaacgcaagggaacggcgcaggatgcacgggctgaaccacgccttcgaccagctgcgcaacgttatcccgtccttcaacaacgacaagaagctgtccaaatatgagaccctacagatggcccagatctacatcaacgctctgtcggagttgctgcagactcccaatgtcggagagcaaccgccgccgcccacagcttcctgcaaaaatgaccaccatcaccttcgcaccgcctcctcctatgaaggaggtgcgggcgcctctgcggtagctggggctcagccagccccgggagggggcccgagacctaccccgcccgggccttgccggactcgcttctcaggcccagcttcctctgggggttactcggtgcagctggacgctttgcacttcccagccttcgaggacagggccctaacagcgatgatggcacagaaggacctgtcgccttcgctgcccgggggcatcctgcagcctgtacaggaggacaacagcaaaacatctcccagatcccacagaagtgacggagagttttccccccactctcattacagtgactctgatgaggccagtctcgagccatctgctggagacatgagggctgccaacctttggccaagccctcttgtgattaagcacactaagaagaatagccctgccttgtccttgacagctgaccagatggtcagtgccttgttggatgctgaaccgcccatgatctattctgaatatgatccttctagacccttcagtgaagcctcaatgatgggcttattgaccaacctagcagatagggagctggttcatatgatcaactgggcaaagagagtgccaggctttggggacttgaatctccatgatcaggtccaccttctcgagtgtgcctggctggagattctgatgattggtctcgtctggcgctccatggaacacccggggaagctcctgtttgctcctaacttgctcctggacaggaatcaaggtaaatgtgtggaaggcatggtggagatctttgacatgttgcttgctacgtcaagtcggttccgcatgatgaacctgcagggtgaagagtttgtgtgcctcaaatccatcattttgcttaattccggagtgtacacgtttctgtccagcaccttgaagtctctggaagagaaggaccacatccaccgtgtcctggacaagatcacagacactttgatccacctgatggccaaagctggcctgactctgcagcagcagcatcgccgcctagctcagctccttctcattctttcccatatccggcatatgagtaacaaacgcatggagcatctctacaacatgaaatgcaagaacgtggtacccctctatgacctgctcctggagatgttggatgcccaccgccttcatgccccagccagtcgcatgggagtgcccccagaggagcccagccagacccagctggccaccaccagctccacttcagcacattccttacaaacctactacatacccccggaagcagagggcttccccaacacgatctcaggatctggttcaggagccacaaccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtctatctatatggccaagaagcccgtgcagctgcccggctactactacgtggactccaagctggacatcacctcccacaacgaggactacaccatcgtggagcagtacgagcgcgccgagggccgccaccacctgttcctgtaggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 16 - DNA, 3125 bp, Artificial Sequence
  Description of Artificial Sequence Synthetic polynucleotide
gcgcatggtaccgccaccatggactacaaagacgatgatgataaagattacaaagatgacgatgacaaggggtcccgcctgctgcatgcagaagagtgggctgaggtaaaagagttgggggaccaccatcgccatccccagccgcaccacgtcccgccgctgacgccacagccacctgctaccctgcaggcgagagaccttcccgtctacccggcagaactgtccctcctggatagcaccgacccacgcgcctggctgactcccactttgcagggcctctgcacggcacgcgccgcccagtatctgctgcattctcccgagctgggtgcctccgaggccgcggcgccccgggacgaggctgacagccagggtgagctggtaaggagaagcggctgtggcggcctcagcaagagccccgggcccgtcaaagtacgggaacagctgtgcaagctgaagggtggggttgtagtggacgagcttggctgcagccgccagcgagccccttccagcaaacaggtgaatggggtacagaagcaaaggaggctggcagcaaacgcaagggaacggcgcaggatgcacgggctgaaccacgccttcgaccagctgcgcaacgttatcccgtccttcaacaacgacaagaagctgtccaaatatgagaccctacagatggcccagatctacatcaacgctctgtcggagttgctgcagactcccaatgtcggagagcaaccgccgccgcccacagcttcctgcaaaaatgaccaccatcaccttcgcaccgcctcctcctatgaaggaggtgcgggcgcctctgcggtagctggggctcagccagccccgggagggggcccgagacctaccccgcccgggccttgccggactcgcttctcaggcccagcttcctctgggggttactcggtgcagctggacgctttgcacttcccagccttcgaggacagggccctaacagcgatgatggcacagaaggacctgtcgccttcgctgcccgggggcatcctgcagcctgtacaggaggacaacagcaaaacatctcccagatcccacagaagtgacggagagttttccccccactctcattacagtgactctgatgaggccagtctcgagccatccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgatctcgagccatctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagcttgagcggccgcatgcgc

SEQ ID NO: 17 - protein, 120 aa, Homo sapiens
MDIKNSPSSLNSPSSYNCSQSILPLEHGSIYIPSSYVDSHHEYPAMTFYSPAVMNYSIPSMDIKNSPSSLNSPSSYNCSQSILPLEHGSIYIPSSYVDSHHEYPAMTFYSPAVMNYSIPS

SEQ ID NO: 18 - protein, 60 aa, Homo sapiens
MAGHLASDFAFSPPPGGGGDGPGGPEPGWVDPRTWLSFQGPPGGPGIGPGVGPGSEVWGI

(19) United States
(12) Patent Application Publication
(10) Pub. No.: US 2014/0134136 A1
(43) Pub. Date: May 15, 2014

(54) COMPOSITIONS AND METHODS FOR AUDITORY THERAPY

(71) Applicants: MASSACHUSETTS EYE AND EAR INFIRMARY, Boston, MA (US); Genesys Research Institute, Brighton, MA (US)

(72) Inventors: Mark A. Parker, Brighton, MA (US); Albert Edge, Boston, MA (US)

(73) Assignees: MASSACHUSETTS EYE AND EAR INFIRMARY, Boston, MA (US); GENESYS RESEARCH INSTITUTE, Brighton, MA (US)

(21) Appl. No.: 14/070,763

(22) Filed: Nov. 4, 2013

Related U.S. Application Data
(60) Provisional application No. 61/722,094, filed on Nov. 2, 2012.

(51) Int. Cl. C07K 14/72; C12N 15/62

(52) U.S. Cl. CPC C07K 14/721; C12N 15/62
USPC 424/93.6; 536/23.5; 514/44 R; 424/93.7; 435/320.1; 435/235.1; 435/325; 435/366; 604/501

(57) ABSTRACT

The invention provides compositions for inducing expression in hair cells, and provides methods of using these compositions for modulating cochlear expression. Such compositions are further useful in treatment of sensorineural hearing loss, e.g., increasing proliferation or survival of mechanosensory hair cells.

### RELATED APPLICATIONS

This application claims the benefit of priority under 35 U.S.C. §119(e) to U.S. Provisional Application No. 61/722,094, filed Nov. 2, 2012, which is incorporated herein by reference in its entirety.

### STATEMENT OF RIGHTS TO INVENTIONS MADE UNDER FEDERALLY SPONSORED RESEARCH

This work was supported by the following grant from the National Institutes of Health, Grant No: R03DC010065. The government has certain rights in the invention.

### BACKGROUND OF THE INVENTION

As many as three persons out of every 1,000 in the United States are born deaf or exhibit a hearing loss (NIDCD 2008). However, as a person advances in age, their chance of developing a hearing loss increases. It is estimated that 17% of adults in the United States exhibit some degree of hearing loss (NIDCD 2003). Thirty percent of people over the age of 65 exhibit a hearing loss, and this percentage increases to 47% of persons 75 and older (NIDCD 2008). Regardless of the etiology, the death or dysfunction of mechanosensory hair cells located within the organ of Corti of the cochlea is the primary cause of sensorineural deafness. For example, overexposure to noise or aminoglycoside antibiotics, results in hair cell loss and subsequent sensorineural hearing loss (SNHL).

Current treatments for SNHL are been based on electronic technologies such as amplification of the speech signal using hearing aids and electrical stimulation of the surviving spiral ganglion neurons using cochlear implants. Many cases of postlingual SNHL, such as presbycusis, are treated using amplification provided by hearing aids. While hearing aids provide a benefit for those who exhibit mild to moderate hearing loss and exhibit a good ability to discriminate speech stimuli, there is a significant population with severe-to-profound SNHL who receive minimal communicative benefit from hearing aids.

Severe-to-profound SNHL is commonly treated using cochlear implants, which directly stimulate the surviving auditory nerve fibers. In terms of speech recognition in quiet environments, cochlear implants have proven to be more effective than hearing aids for those who exhibit severe-to-profound SNHL. However, their use does not restore normal hearing. Implant users in general also perform poorly in the presence of background noise, a difficulty which is exacerbated when the competing noise consists of speech stimuli. Cochlear implant recipients also exhibit a relatively poor ability to localize sound, an effect that is particularly noted in patients with unilateral implants. Finally, cochlear implant recipients exhibit poor pitch perception, which interferes with perception of music, and poor representation of tonal languages such as Punjabi of India, and Chinese languages such as Mandarin, Cantonese, and Taiwanese. Therefore, a significant population of deaf people who would communicate using tonal languages are underserved with current implant technologies. Accordingly, new methods of treatment of SNHL are urgently required.

The basic helix-loop-helix transcription factor atonal-1 (Atoh1 or Math1) is involved in mammalian hair cell development. Expression of Atoh1 in cochlear cells is both important and sufficient for hair cell genesis in the ear. Experiments indicate that forced expression of Atoh1 can be used to regenerate lost hair cells, although the mechanism of Atoh1-induced hair cell regeneration has not been fully characterized. One drawback to forced expression is a lack of regulation of gene expression in transfected cells. Rather than using a constitutive expression system, where Atoh1 is expressed continually and at abnormally elevated levels, it would be useful and desirable to have an inducible system for modulating Atoh1 expression, for example in a cochlear environment. The inability to modulate Atoh1 remains an obstacle to on-going research in hair cells and the development of therapeutics for hair regeneration.

### SUMMARY OF THE INVENTION

As described below, the present invention features compositions and methods for modulating or inducing cochlear expression involving the use of a modified Atoh1 transcription factor fused to an estrogen receptor that localizes to the nucleus when contacted with an estrogen receptor ligand (e.g., 4-hydroxy tamoxifen sulfate), thereby activating expression of genes having Atoh1 responsive promoters.

In one aspect the invention provides an isolated nucleic acid having a sequence that encodes a polypeptide having Atonal homolog 1 (Atoh1) or fragment thereof operably linked to an estrogen receptor (ER) or fragment thereof, where the Atoh1 or fragment thereof can bind nucleic acid and can activate transcription, and where the ER or fragment thereof can bind an ER ligand.

In another aspect, the invention provides a method for treating or preventing hearing loss (e.g. sensorineural hearing loss) in an individual, involving administering to an individual in need thereof a pharmacologically effective dose of a pharmaceutical composition containing a nucleic acid having a sequence that encodes a polypeptide having Atonal homolog 1 (Atoh1) or fragment thereof operably linked to an estrogen receptor (ER) or fragment thereof, where the Atoh1 or fragment thereof can bind nucleic acid and can activate transcription, and where the ER or fragment thereof can bind an ER ligand.

In yet another aspect, the invention provides a method for enhancing hair cell growth, maintenance, survival, or proliferation, involving administering to a hair cell a nucleic acid having a sequence that encodes a polypeptide having Atonal homolog 1 (Atoh1) or fragment thereof operably linked to an estrogen receptor (ER) or fragment thereof, where the Atoh1 or fragment thereof can bind nucleic acid and can activate transcription, and where the ER or fragment thereof can bind an ER ligand.

In still another aspect, the invention provides a method for reducing hair cell death or apoptosis, involving administering to a hair cell a nucleic acid having a sequence that encodes a polypeptide having Atonal homolog 1 (Atoh1) or fragment thereof operably linked to an estrogen receptor (ER) or fragment thereof, where the Atoh1 or fragment thereof can bind nucleic acid and can activate transcription, and where the ER or fragment thereof can bind an ER ligand.

In another aspect, the invention provides a method for treating or preventing neoplasia in an individual, involving administering to an individual in need thereof a pharmacologically effective dose of a pharmaceutical composition containing a nucleic acid having a sequence that encodes a polypeptide having Atonal homolog 1 (Atoh1) or fragment thereof operably linked to an estrogen receptor (ER) or fragment thereof, where the Atoh1 or fragment thereof can bind nucleic acid and can activate transcription, and where the ER or fragment thereof can bind an ER ligand.

In yet another aspect, the invention provides a method for decreasing neoplastic cell growth, maintenance, survival, or proliferation, involving administering to a neoplastic cell a nucleic acid having a sequence that encodes a polypeptide having Atonal homolog 1 (Atoh1) or fragment thereof operably linked to an estrogen receptor (ER) or fragment thereof, where the Atoh1 or fragment thereof can bind nucleic acid and can activate transcription, and where the ER or fragment thereof can bind an ER ligand.

In still another aspect, the invention provides a method for increasing neoplastic cell death or apoptosis, involving administering to a neoplastic cell a nucleic acid having a sequence that encodes a polypeptide having Atonal homolog 1 (Atoh1) or fragment thereof operably linked to an estrogen receptor (ER) or fragment thereof, where the Atoh1 or fragment thereof can bind nucleic acid and can activate transcription, and where the ER or fragment thereof can bind an ER ligand.

In a related aspect, the invention provides a polypeptide having Atonal homolog 1 (Atoh1) or fragment thereof operably linked to an estrogen receptor (ER) or fragment thereof, where the Atoh1 or fragment thereof can bind nucleic acid and can activate transcription, and where the ER or fragment thereof can bind an ER ligand. In a further aspect, the invention provides a pharmaceutical composition containing a polypeptide according to any of the aspects described herein.

In another related aspect, the invention provides a vector having a nucleic acid according to any of the aspects described herein. In a further aspect, the invention provides a virus containing a vector according to any of the aspects described herein.

In still another related aspect, the invention provides a host cell containing a vector according to any of the aspects described herein. In a further aspect, the invention provides a xenograft including a cell according to any of the aspects described herein.

In various embodiments of the aspects described herein, the Atoh1 or fragment thereof and the ER or fragment thereof are linked by a linker. In various embodiments of the aspects described herein, the ER or fragment thereof is operatively linked to the C-terminus of the Atoh1 or fragment thereof. In some cases, the polypeptide does not comprise a reporter construct. In other cases, the polypeptide further includes a reporter selected from the group consisting of DsRed, GFP, RFP, BFP, CFP, and YFP. In various embodiments, the reporter is linked to the Atoh1 or fragment thereof or the ER or fragment thereof by a linker. In certain embodiments, the reporter is operatively linked to the C-terminus of the ER or fragment thereof. In various embodiments of the aspects described herein, the polypeptide is expressed from a vector that is administered to the subject. In various embodiments, the polypeptide is expressed in a host cell that is administered to the subject.

In various embodiments of the aspects described herein, the ER or fragment thereof has been modified to limits endogenous 17b-estradiol binding at physiological concentrations. In various embodiments of the aspects described herein, the ER ligand is selected from the group consisting of 4-hydroxy Tamoxifen, Tamoxifen, and estrogen. In various embodiments of the aspects described herein, the polypeptide localizes to the nucleus when contacted with an ER ligand.

In various embodiments of the aspects described herein, the vector is an expression vector suitable for expression in a mammalian cell. In various embodiments, the vector includes an enhancer or promoter. The construct can easily be placed under control of different promoters to confer cell specific expression. For example, a polynucleotide encoding a therapeutic or reporter protein, variant, or a fragment thereof, can be cloned into a retroviral vector, and expression can be driven from its endogenous promoter, from the retroviral long terminal repeat, or from a promoter specific for a target cell type of interest. In some cases, the vector includes an enhancer or promoter of a gene selected from the group consisting of Glial fibrillary acidic protein (GFAP), SRY (sex determining region Y)-box 2 (Sox2), Prospero homeobox protein 1 (prox1), and Transforming Growth Factor β-activated Kinase 1 (TAK1).

In various embodiments of the aspects described herein, the vector is in a virus (e.g., that is administered to the subject). In various embodiments of the aspects described herein, the virus is one or more of a cytomegaloviris, lentivirus, adenovirus, retrovirus, adeno-associated virus, herpesvirus, vaccinia virus, or polyoma virus.

In various embodiments of the aspects described herein, the vector is in a host cell (e.g., that is administered to the subject). In various embodiments of the aspects described herein, the cell is in vitro, in vivo, or ex vivo. In various embodiments of the aspects described herein, the cell is a mammalian cell or human cell. In various embodiments of the aspects described herein, the cell is derived from a tumor or immortalized cell line. In various embodiments of the aspects described herein, the cell is a hair cell or cochlear cell. In various embodiments of the aspects described herein, the host cell is in a xenograft that is administered to the subject.

In various embodiments of the aspects described herein, the hearing loss is sensorineural hearing loss. In various embodiments of the aspects described herein, the hair cell is a cochlear cell. In various embodiments of the aspects described herein, the neoplasia or neoplastic cell type is selected from the group consisting of intestinal cancer, colorectal cancer, skin cancer, brain cancers such as gliomas and medulloblasomas and neuroendocrine cancers.

The invention provides compositions and methods that provide for the localization of Atoh1 to the nucleus and expression of genes regulated by the Atoh1 transcription factor. Compositions and articles defined by the invention were isolated or otherwise manufactured in connection with the examples provided below. Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide (e.g., reporter) as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “Atonal Homolog 1”, “Atoh1”, “Atoh1 protein”, or “Atoh1” as used herein, shall refer to a polypeptide having an amino acid sequence at least 85, 90, 95, 96, 97, 98, 99 or 100% identical to GenBank Accession No. NP_{—}005163. Atoh1 is suitable for use with the present invention include Atoh1 and fragments thereof that bind enhancer/promoter sequences and activate transcription. An exemplary Atoh1 of the invention is human Atoh1.

By “binding to” a molecule is meant having a physicochemical affinity for that molecule.

As used herein, “cassette” or “reporter cassette” means a DNA sequence capable of directing expression of a nucleotide sequence in a cell. In one embodiment, a cassette comprises a promoter operably linked to a nucleotide sequence of interest that is optionally operably linked to termination signals and/or other regulatory elements. A cassette may also comprise sequences required for proper translation of the nucleotide sequence. The expression cassette comprising the nucleotide sequence of interest may be chimeric, meaning that at least one of its components is heterologous with respect to at least one of its other components. For example, in certain embodiments of the invention an Atoh1 transcription factor is operably linked to an estrogen receptor polypeptide and a detectable reporter (e.g., DsRed) to form a fusion polypeptide. An expression cassette may be assembled entirely extracellularly (e.g., by recombinant cloning techniques). The expression of the nucleotide sequence in the expression cassette may be under the control of a constitutive promoter or an inducible promoter which initiates transcription only when the host cell is exposed to some particular stimulus. In the case of a multicellular organism, expression of a reporter in the cassette can be specific to a particular microenvironment, tissue, organ, or stage of development.

By “compound” is meant any small molecule chemical compound, antibody, nucleic acid molecule, or polypeptide, or fragments thereof.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell (e.g., hair cell), tissue (e.g., cochlear), or organ (e.g., ear).

By “enhancer”, as used herein, refers to a regulatory nucleic acid sequence, which can function in either orientation and in any location with respect to a promoter, to modulate (e.g., increase) the effect of a promoter (e.g., to increase transcription levels).

By “Estrogen receptor”, “ER”, “ER protein”, or “ER” as used herein, shall refer to a polypeptide having an amino acid sequence at least 85, 90, 95, 96, 97, 98, 99 or 100% identical to GenBank Accession No. NP_{—}000116 [Estrogen receptor alpha] or NP_{—}001035365 [Estrogen receptor beta]. Estrogen receptor polypeptides suitable for use with the present invention include Estrogen receptor and fragments thereof that contain the ligand binding domain. An exemplary Estrogen receptor of the invention is a human Estrogen receptor variant modified to limit endogenous 17b-estradiol binding at physiological concentrations (Danielian et al., 1998; Danielian et al., 1993). The mutation of the glycine at position 525 and the methionine and/or serine at positions 521/522 virtually abolished the ability of the receptor to bind estradiol and stimulate transcription (Danielian et al., 1993).

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Fusion polypeptide” or “fusion protein”, as used herein, shall mean a polypeptide comprising two or more different polypeptides or active fragments thereof that are not naturally present in the same polypeptide. Generally, the two or more different polypeptides are linked together covalently, e.g., chemically linked or fused in frame by a peptide bond.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

“Linker”, as used herein, shall mean a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple Atoh1 to ER ligand binding domain). In the fusion polypeptides of the invention, linker sequences were designed to translate into multiple amino acid sequences to provide an increased degree of freedom for the subunits of the fusion protein. Exemplary linker sequences include CTCGAGCCATCTGCTGGAGACATG (SEQ ID NO: 1, which was used to link the C-terminus of Atoh1 to the N-terminus of an Estrogen receptor binding domain) and TCAGGATCTGGTTCAGGA (SEQ ID NO: 2, which was used to link the C-terminus of an Estrogen receptor binding domain to the N-terminus of DsRed).

By “marker” is meant any protein or polynucleotide having an alteration in expression level or activity that is associated with a disease or disorder.

“Operatively linked”, as used herein, shall mean the linking of two or more biomolecules so that the biological functions, activities, and/or structure associated with the biomolecules are at least retained. In reference to polypeptides, the term means that the linking of two or more polypeptides results in a fusion polypeptide that retains at least some of the respective individual activities of each polypeptide component. The two or more polypeptides may be linked directly or via a linker. In reference to nucleic acids, the term means that a first polynucleotide is positioned adjacent to a second polynucleotide that directs transcription of the first polynucleotide when appropriate molecules (e.g., transcriptional activator proteins) are bound to the second polynucleotide.

By “neoplasia” is meant a disease or disorder characterized by excess proliferation or reduced apoptosis. Illustrative neoplasms for which the invention can be used include, but are not limited to leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

As used herein, “obtaining” as in “obtaining an agent” includes synthesizing, purchasing, or otherwise acquiring the agent.

By “promoter” is meant a polynucleotide sufficient to direct transcription.

By “protein” or “polypeptide” or “peptide” is meant any chain of more than two natural or unnatural amino acids, regardless of post-translational modification (e.g., glycosylation or phosphorylation), constituting all or part of a naturally-occurring or non-naturally occurring polypeptide or peptide, as is described herein.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “reduce” or “increase” is meant to alter negatively or positively, respectively, by at least 5%. An alteration may be by 5%, 10%, 25%, 30%, 50%, 75%, or even by 100%.

By “reference” is meant a standard or control condition. In one embodiment, the effect of an agent on a cell is compared to the effect of the agent on a control cell.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “reporter” is meant a molecule (e.g., a polypeptide) that is detectable or has a detectable property (e.g., fluorescence). In the reporter cassettes of the invention, the coding region encodes a reporter. A “detectable reporter” is a polypeptide that comprises a moiety that renders it detectable, via any means, including spectroscopic, photochemical (e.g., luciferase, GFP), biochemical, immunochemical, or chemical means. Detectable reporters of the invention include for example GFP, evoglow, mCherry, and RFP.

By “regulatory element” or “regulatory sequence” is meant a nucleic acid which, when operably linked to a polynucleotide, modulates transcription and/or expression levels of the polynucleotide in a cell. Genetic regulatory elements of the present invention may include promoters, enhancers, insulators, or a combination thereof, as well as other cis-acting sequences involved in the binding of transcription factors. Regulatory elements include both positive and negative regulators of transcription.

As used herein, the terms “selectable marker” or “selectable marker gene” is meant a nucleic acid sequence that confers a particular phenotype upon a cell. In one embodiment, the selectable marker confers resistance to an antibiotic or drug. In another embodiment, the selectable marker provides an enzymatic activity that confers the ability to grow in medium lacking a nutrient. Antibiotic selectable markers used in the vectors of the invention include resistance genes for puromycin, hygromycin, or neomycin. When a host cell must express a selectable marker to grow in selective medium, the marker is said to be a positive selectable marker (e.g., antibiotic resistance genes which confer the ability to grow in the presence of the appropriate antibiotic). Selectable markers can also be used to select against host cells containing a particular gene; selectable markers used in this manner are referred to as negative selectable markers.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule.

By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .mu·g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “Sensorineural hearing loss” or “SNHL” is meant hearing loss caused by death or dysfunction of cochlear cells, including mechanosensory hair cells.

By “specifically binds” is meant a compound or antibody that recognizes and binds a polypeptide of the invention, but which does not substantially recognize and bind other molecules in a sample.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰° indicating a closely related sequence.

By “subject” is meant a mammal, including, but not limited to, a human or non-human mammal, such as a bovine, equine, canine, ovine, feline, or rodent.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1A-FIG. 1E are schematics depicting constructs used to generate an inducible Atoh1 fusion protein. FIG. 1A depicts the construct termed Flag-Atoh1-ER-DsRed. In order to measure the translocation of the fusion protein between the cytoplasm and cytosol, a construct was engineered to include the DsRed transgene. To aid in the detection of Atoh1 (Atonal homolog 1), several experimental constructs were engineered to include one or two consecutive flag-tagged sequences (Vallier, Mancip et al. 2001). To obtain the tamoxifen sensitive Atoh1 construct, PCR cloning primers were designed so that 1) an EcoRI site was placed on the 5′ end and a Kozac sequence (CACC) was placed upstream of the Atoh1 start codon; 2) the Atoh1 stop codon (TAG) was deleted; 3) the flag tagged Atoh1 sequence was linked to an Estrogen Receptor (ER) sequence by the sequence CTCGAGCCATCTGCTGGAGACATG (SEQ ID NO: 1) encoding a polypeptide linker; 4) the ER stop codon (TAG) was deleted; 5) the ER sequence was linked to a DsRed sequence by the sequence TCAGGATCTGGTTCAGGA (SEQ ID NO: 2) encoding a polypeptide linker; and 6) a Not I site was included on the 3′ end. The linker sequences were designed to translate into multiple proline sequences, which provide an increased degree of freedom for the fusion protein subunits. The insert for the ER construct was amplified using a 2-step PCR from template DNA that has been mutated to limit endogenous 17b-estradiol binding at physiological concentrations (Danielian, White et al. 1993; Danielian, Muccino et al. 1998) and was obtained from template DNA provided by A. McMahon (Harvard Medical School) and DsRed DNA was obtained from a commercial vector (Clonetech). This construct was generated through subclonings into pcDNATM3.1(+) vector. The nucleic acid sequence of a vector encoding Flag-Atoh1-ER-DsRed is provided below. FIG. 1B depicts the construct termed DsRed-ER, which was used as a negative control. To make the negative control DsRed-ER construct, PCR cloning primers were designed so that 1) an EcoR I site was placed on the 5′ end and a Kozac sequence (CACC) was placed upstream of the DsRed start codon; 2) the stop codon (TAG) for DsRed was deleted; 3) DsRed was linked to ER by the sequence TCAGGATCTGGTTCAGGATCCATG (SEQ ID NO: 3) encoding a polypeptide linker; and 4) a Not1 site was cloned onto the 3′ end. FIG. 1C depicts the construct termed Flag-Atoh1-ER. This construct encodes a fusion protein that binds to and activates the Atoh1 promoter/enhancer. The nucleic acid sequence of a vector encoding Flag-Atoh1-ER is provided below. Atoh1 promoter/enhancer binding, Atoh1 mRNA transcription, and Atoh1 protein translation are on average higher than, but not significantly different from, those of the flag-Atoh1-ER-DsRed construct. FIG. 1D depicts the construct termed Atoh1-ER. This construct is identical to the Flag-Atoh1-ER construct with the exclusion of the flag tag sequences. This construct exhibits activities that are not significantly different than the Flag-Atoh1-ER-DsRed or Flag-Atoh1-ER construct. FIG. 1E depicts the construct termed TAK1p-Atoh1-ER. This construct is identical to the Atoh1-ER construct with the exception that its expression is placed under control of the TAK1 promoter/enhancer. Therefore expression of this transgene will occur in cell types that express endogenous TAK1. Each of these transgenes may be placed under control of this promoter and there is no significant difference in expression between construct expression.

FIG. 2 depicts that 4-hydroxy tamoxifen sulfate (4OHT) induced localization of Atoh1-ER-DsRed to the nucleus in a dose-dependent manner. HEK cells were transfected with Atoh1-ER-DsRed and incubated in graded doses of 4OHT. In the absence of 4OHT, the Atoh1-ER-DsRed fusion protein is sequestered to the cytoplasm. Contacting cells expressing Atoh1-ER-DsRed with tamoxifen increased DsRed fluorescence in the nucleus (left panel, 0 nM Tamoxifen; middle panel, 1 nM Tamoxifen; 1 μM Tamoxifen). Increasing concentrations of 4OHT result in a nuclear localization of the Atoh1-ER-DsRed fusion protein. Nuclear fractionation revealed a 4OHT-dependent increase in DsRed fluorescence in isolated nuclei. The minimum effective dose was empirically determined to be 1 nM tamoxifen for 2-7 days. A graph of DsRed fluorescence against increasing Tamoxifen concentration indicated 1 μM demonstrated increased fluorescence compared to higher (100 μM) and lower doses (1 nM). The highest dose (100 mM) produced cytotoxic effects after 2 days in culture.

FIGS. 3A and 3B depict the determination of optimal 4-hydroxy tamoxifen sulfate (4OHT) concentration and incubation time for nuclear localization. Atoh1-ER-DsRed was electroporated into cochlear spheres generated from ROSA26-GFP mice (green) and incubated with graded doses of 4OHT. FIG. 3A depicts in the absence of 4OHT, the Atoh1-ER-DsRed fusion protein (red) is expressed exclusively in the cytoplasm. FIG. 3B depicts addition of 4OHT (1 nM) results in punctate nuclear localization of the fusion protein after 48 hrs. (DAPI=blue; DsRed fluorescence=red). The Table in the bottom panel depicts dose and temporal effects of 4OHT on nuclear localization. Yellow box highlights optimal conditions (+=nuclear localization in <0.90% of cells; −/+≈50% nuclear localization; −=>0.10% nuclear localization.

FIG. 4 depicts 4-hydroxy tamoxifen sulfate (4OHT) induced activation of the Atoh1 enhancer region in HEK cells that had been stably transfected with a cmv.Atoh1enhancer-luciferase construct. The stably transfected HEK cells were transiently transfected with either the cmv.Atoh1-ER-DsRed construct or a cmv.DsRed-ER control construct. Cells were incubated for 72 hrs. in increasing doses of 4OHT, then lysed and subjected to luciferase assay (Invitrogen). All cells were also co-transfected with Renilla transfection controls.

FIG. 5 depicts that 4-hydroxy tamoxifen sulfate (4OHT) induced Atoh1 mRNA expression in HEK cells transiently transfected with the Atoh1-ER-DsRed construct. Transiently transfected HEK cells were incubated with different doses of 4OHT. RT-PCR suggests that an increase in 4OHT results in an increase in Atoh1 mRNA levels (top panel). Quantitative PCR indicates that increasing doses of 4OHT result in an increase in Atoh1 mRNA expression (bottom panel).

FIG. 6 depicts that 4-hydroxy tamoxifen sulfate (4OHT) induced Atoh1 protein expression in HEK cells transiently transfected with the Atoh1-ER (FMER) construct. Transiently transfected HEK cells were incubated with different doses of 4OHT for 72 hr. Whole cell protein was collected and processed for Western blot analysis. Positive control samples were transfected with a flag-tagged Atoh1 construct under control of a cmv promoter (flagAtoh1) and negative control samples were transfected with the DsRedER construct. Increasing levels of 4OHT resulted in an increase in Atoh1 protein levels in cells transfected with Atoh1-ER.

FIG. 7 depicts that Tamoxifen induced Atoh1 expression. A cmv promoter drives constitutive expression of the Atoh1-ER-DsRed fusion protein, which is sequestered in the cytosol by HSP90 (left panel). Without being bound to a particular theory, 4-hydroxytamoxofen (4-OHT) competes with HSP90 and allows the Atoh1-ER-DsRed fusion protein to translocate to the nucleus (right panel) where it binds to the endogenous Atoh1 enhancer/promoter region and expresses endogenous Atoh1 in a feed-forward mechanism. The Atoh1-ER-DsRed construct can easily be placed under control of different promoters to confer cell specific expression.

FIG. 8 depicts that 4OHT induced Atoh1 expression in cultured organs of Corti. OC1 cells were transfected with the Atoh1-ER-DsRed construct, cultured as floating aggregates in proliferating conditions (33° C.) for 3 days, and then cultured for 3 days in differentiating conditions (39° C.) in either the presence or absence of 4OHT. There was no significant difference in transfection efficiencies between these 2 groups (DsRed bars on chart). However, culturing these spheres in tamoxifen resulted in a significant increase in myosin 7a positive cells within the spheres.

FIG. 9 depicts temporal, quantitative, and cell-specific up-regulation of Atoh1 in the cochlea. The TAK1p-Atoh1-ER construct can be loaded into any vector such as a virus (top panel), which can be injected into the scala media (middle panel). After hair cell damage, supporting cells can transdifferentiate into hair cells by systemic 4OHT (bottom panel).

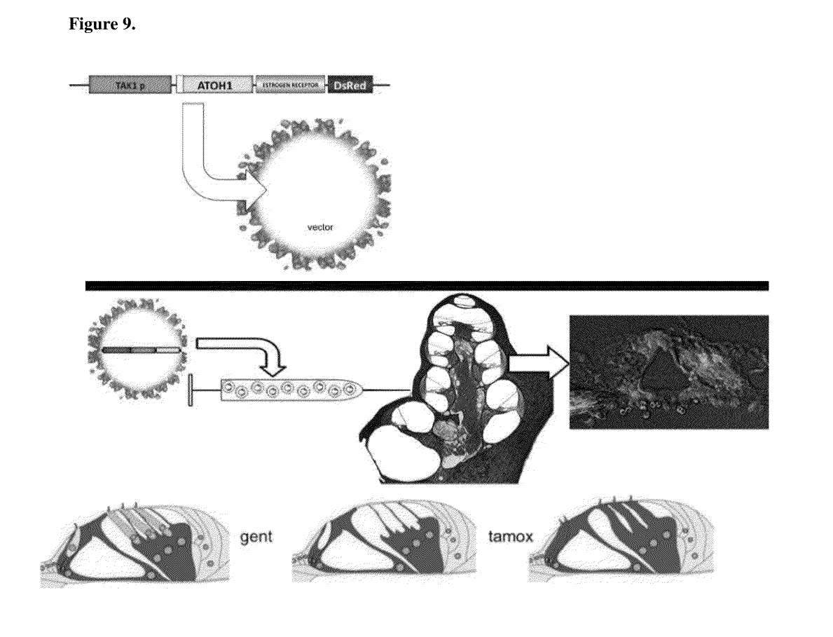

### DETAILED DESCRIPTION OF THE INVENTION

The invention features compositions for inducibly localizing Atoh1 to the nucleus and regulating Atoh1-mediated expression, and provides methods of using these compositions for growing and/or regenerating hair cells. Such compositions are further useful in methods of treating sensorineural hearing loss and neoplasia (e.g., colon cancer, breast, and skin cancer).

As reported in more detail below, the present application provides expression vectors encoding a fusion polypeptide comprising Atoh1 and an ER ligand binding domain. In some embodiments the fusion polypeptide further comprises a reporter (e.g., DsRed). The invention further provides methods for localizing the fusion polypeptide to the nucleus. Localization of Atoh1 to the nucleus results in expression of genes regulated by Atoh1 responsive enhancers/promoters, including Atoh1 itself.

An inducible model was developed that allows for the conditional expression of Atoh1 in the organ of Corti. An Atoh1 gene was generated having a C-terminal fusion to the estrogen receptor (ER) and a reporter protein (DS-Red) to increase Atoh1 expression in in a dose-dependent manner by the addition of tamoxifen to cultured cells or to the cochlear environment. HEK cells transfected with this construct exhibited constitutive expression of the Atoh1-ER-DsRed fusion protein in the cytoplasm, where it is rendered quiescent. The addition of tamoxifen to the transfected cells resulted in a dose-dependent localization of the Atoh1-ER-DsRed fusion protein to the nucleus. Removal of tamoxifen from the culture media resulted in a cytoplasmic localization of the fusion protein within 2 weeks. Because Atoh1 acts as an autoregulatory transcription factor that positively regulates its own transcription, increasing concentrations of tamoxifen induced a dose-dependent increase in binding to the enhancer/promoter region of the Atoh1 gene as measured by a luciferase assay, and tamoxifen increased the expression of Atoh1 in a dose-dependent manner, as determined by both RT-PCR and qPCR. Organs of Corti electroporated with this construct expressed supernumerary hair cells when exposed to 1 μM tamoxifen. These data indicate that the Atoh1-ER-DsRed fusion protein may be used for time and dose-dependent regulation of Atoh1 expression. Thus, the invention is based, at least in part, on the observation that an Atoh1-ER-DsRed fusion polypeptide localizes to the nucleus when contacted with 4-hydroxy tamoxifen. When the Atoh1-ER-DsRed fusion polypeptide localized to the nucleus, it was able to activate transcription of Atoh1 and myosin 7a. The nuclear localization of Atoh1-ER fusion proteins is useful for activating cochlear specific expression and for modulating Atoh1 tumor suppressor activity.

### Atonal Homolog 1 (Atoh1)

One of the definitive genes for hair cell development is the mammalian homolog of the basic helix-loop-helix transcription factor atonal-1 (Atoh1). Atoh1 also displays an anti-oncogenic function or tumor suppressor function (Bossuyt et al., 2009). Expression of Atoh1 in cochlear cells is both required and sufficient for hair cell genesis (Bermingham, Hassan et al. 1999). Cells within the developing organ of Corti that express Atoh1 will differentiate into hair cells (Helms, Abney et al. 2000), and Atoh1 is one of the earliest markers of hair cell differentiation. Atoh1 knock-out mice fail to develop hair cells (Isaka, Ishibashi et al. 1999; Helms, Abney et al. 2000). Supporting cells of the organ of Corti that over-express the pro hair cell gene Atoh1 maintain the potential to develop hair cell characteristics including cilia formation (Zheng and Gao 2000; Kawamoto, Ishimoto et al. 2003; Izumikawa, Minoda et al. 2005), myosin 7a labeling (Zheng and Gao 2000), and proper hair cell function (Kawamoto, Ishimoto et al. 2003). Electroporation of Atoh1 into fetal otocysts (Gubbels, Woessner et al. 2008) and organs of Corti explants resulted in hair cell genesis (Zheng and Gao 2000).

The amino acid sequence of human Atoh1 is provided at NCBI Accession No. NP_{—}005163, which is reproduced below (SEQ ID NO: 4):

| | |
|---|---|
| 1 | msrllhaeew aevkelgdhh rqpqphhlpq pppppqppat lqarehpvyp pelslldstd |
| 61 | prawlaptlq gictaraaqy llhspelgas eaaaprdevd grgelvrrss ggassskspg |
| 121 | pvkvreqlck lkggvvvdel gcsrqrapss kqvngvqkqr rlaanarerr rmhglnhafd |
| 181 | qlrnvipsfn ndkklskyet lqmaqiyina lsellqtpsg geqpppppas cksdhhhlrt |
| 241 | aasyeggagn ataagaqqas ggsqrptppg scrtrfsapa saggysvqld alhfstfeds |
| 301 | altammaqkn lspslpgsil qpvqeenskt sprshrsdge fsphshysds deas |

Adenoviral mediated delivery of Atoh1 into the cochlea resulted in hair cell genesis in cells infected with this virus (Bermingham, Hassan et al. 1999; Zheng and Gao 2000; Kawamoto, Ishimoto et al. 2003). Interestingly, some of these cells exhibited a chimerical hair/supporting cell morphology suggesting that adult supporting cells maintain the potential for transdifferentiation. Importantly, the data indicate that hair cell genesis is possible in the adult mammalian organ of Corti is from this data. Later experiments suggest that Atoh1 infection in adult guinea pig cochleas results in functional recovery as well (Izumikawa, Minoda et al. 2005).

### Estrogen Receptor

Estrogen receptors are a group of proteins found inside cells. They are receptors that are activated by the hormone estrogen (17β-estradiol). Two classes of estrogen receptor exist: ER, which is a member of the nuclear hormone family of intracellular receptors, and the estrogen G protein-coupled receptor GPR30 (GPER), which is a G protein-coupled receptor. The nuclear hormone family of intracellular estrogen receptors are useful in the methods of the invention. Once activated by estrogen, intracellular estrogen receptors localize to the nucleus, where they are able to bind to DNA and regulate the activity of many different genes (i.e., as a DNA-binding transcription factor). However, intracellular estrogen receptors also have additional functions independent of DNA binding.

There are two different forms of the intracellular estrogen receptor, usually referred to as a and β, each encoded by a separate gene (ESR1 and ESR2, respectively). Hormone-activated estrogen receptors form dimers. Because the two forms are coexpressed in many cell types, the receptors may form ERα (aa) or ERβ (ββ) homodimers or ERαβ (αβ) heterodimers. Estrogen receptor alpha and beta show significant overall sequence homology, and both are composed of five domains (listed from the N- to C-terminus; amino acid sequence numbers refer to human ER):(A-F domain). The N-terminal A/B domain is able to transactivate gene transcription in the absence of bound ligand (e.g., the estrogen hormone). While this region is able to activate gene transcription without ligand, this activation is weak and more selective compared to the activation provided by the E domain. The C domain, also known as the DNA-binding domain, binds to estrogen response elements in DNA. The D domain is a hinge region that connects the C and E domains. The E domain contains the ligand binding cavity as well as binding sites for coactivator and corepressor proteins. The E-domain in the presence of bound ligand is able to activate gene transcription. The C-terminal F domain function is not entirely clear and is variable in length.

The amino acid sequence of human Estrogen Receptor α is provided at NCBI Accession No. NP_{—}000116, which is reproduced below (SEQ ID NO: 5):

| | |
|---|---|
| 1 | mtmtlhtkas gmallhqiqg neleplnrpq lkiplerplg evyldsskpa vynypegaay |
| 61 | efnaaaaana qvygqtglpy gpgseaaafg snglggfppl nsyspsplml lhpppqlspf |
| 121 | lqphgqqvpy ylenepsgyt vreagppafy rpnsdnrrqg grerlastnd kgsmamesak |
| 181 | etrycavcnd yasgyhygvw scegckaffk rsiqghndym cpatnqctid knrrkscqac |
| 241 | rlrkcyevgm mkggirkdrr ggrmlkhkrq rddgegrgev gsagdmraan lwpsplmikr |
| 301 | skknslalsl tadqmvsall daeppilyse ydptrpfsea smmglltnla drelvhminw |
| 361 | akrvpgfvdl tlhdqvhlle cawleilmig lvwrsmehpg kllfapnlll drnqgkcveg |
| 421 | mveifdmlla tssrfrmmnl ggeefvclks iillnsgvyt flsstlksle ekdhihrvld |
| 481 | kitdtlihlm akagltlqqq hqrlaqllli lshirhmsnk gmehlysmkc knvvplydll |
| 541 | lemldahrlh aptsrggasv eetdqshlat agstsshslq kyyitgeaeg fpatv |

The amino acid sequence of human Estrogen Receptor β is provided at NCBI Accession No. NP_{—}001035365, which is reproduced below (SEQ ID NO: 6):

| | |
|---|---|
| 1 | mdiknspssl nspssyncsq silplehgsi yipssyvdsh heypamtfys pavmnysips |
| 61 | nvtnleggpg rqttspnvlw ptpghlsplv vhrqlshlya epqkspwcea rslehtlpvn |
| 121 | retlkrkvsg nrcaspvtgp gskrdahfca vcsdyasgyh ygvwscegck affkrsiqgh |
| 181 | ndyicpatnq ctidknrrks cqacrlrkcy evgmvkcgsr rercgyrlvr rqrsadeqlh |
| 241 | cagkakrsgg haprvrelll dalspeqlvl tlleaepphv lisrpsapft easmmmsltk |
| 301 | ladkelvhmi swakkipgfv elslfdqvrl lescwmevlm mglmwrsidh pgklifapdl |
| 361 | vldrdegkcv egileifdml lattsrfrel klqhkeylcv kamillnssm yplvtatqda |
| 421 | dssrklahll navtdalvwv iaksgissqq qsmrlanllm llshvrhara ekasqtltsf |
| 481 | gmkmetllpe atmeq |

An exemplary nucleic acid sequence encoding an exemplary human Estrogen Receptor is provided below (SEQ ID NO: 7):

| | |
|---|---|
| 1 | mdiknspssl nspssyncsq silplehgsi yipssyvdsh heypamtfys pavmnysips |
| 61 | mdiknspssl nspssyncsq silplehgsi yipssyvdsh heypamtfys pavmnysips |
| 121 | ttaattaaac tagtcttaag aagcttgaat tccaccATGT CCAATTTACT GACCGTACAC |
| 181 | CAAAATTTGC CTGCATTACC GGTCGATGCA ACGAGTGATG AGGTTCGCAA GAACCTGATG |
| 241 | GACATGTTCA GGGATCGCCA GGCGTTTTCT GAGCATACCT GGAAAATGCT TCTGTCCGTT |
| 301 | TGCCGGTCGT GGGCGGCATG GTGCAAGTTG AATAACCGGA AATGGTTTCC CGCAGAACCT |
| 361 | GAAGATGTTC GCGATTATCT TCTATATCTT CAGGCGCGCG GTCTGGCAGT AAAAACTATC |
| 421 | CAGCAACATT TGGGCCAGCT AAACATGCTT CATCGTCGGT CCGGGCTGCC ACGACCAAGT |
| 481 | GACAGCAATG CTGTTTCACT GGTTATGCGG CGGATCCGAA AAGAAAACGT TGATGCCGGT |
| 541 | GAACGTGCAA AACAGGCTCT AGCGTTCGAA CGCACTGATT TCGACCAGGT TCGTTCACTC |
| 601 | ATGGAAAATA GCGATCGCTG CCAGGATATA CGTAATCTGG CATTTCTGGG GATTGCTTAT |
| 661 | AACACCCTGT TACGTATAGC CGAAATTGCC AGGATCAGGG TTAAAGATAT CTCACGTACT |
| 721 | GACGGTGGGA GAATGTTAAT CCATATTGGC AGAACGAAAA CGCTGGTTAG CACCGCAGGT |
| 781 | GTAGAGAAGG CACTTAGCCT GGGGGTAACT AAACTGGTCG AGCGATGGAT TTCCGTCTCT |
| 841 | GGTGTAGCTG ATGATCCGAA TAACTACCTG TTTTGCCGGG TCAGAAAAAA TGGTGTTGCC |
| 901 | GCGCCATCTG CCACCAGCCA GCTATCAACT CGCGCCCTGG AAGGGATTTT TGAAGCAACT |
| 961 | CATCGATTGA TTTACGGCGC TAAGGATGAC TCTGGTCAGA GATACCTGGC CTGGTCTGGA |
| 1021 | CACAGTGCCC GTGTCGGAGC CGCGCGAGAT ATGGCCCGCG CTGGAGTTTC AATACCGGAG |
| 1081 | ATCATGCAAG CTGGTGGCTG GACCAATGTA AATATTGTCA TGAACTATAT CCGTAACCTG |
| 1141 | GATAGTGAAA CAGGGGCAAT GGTGCGCCTG CTGGAAGATG GCGATctcga gccaTCTGCT |
| 1201 | GGAGACATGA GAGCTGCCAA CCTTTGGCCA AGCCCGCTCA TGATCAAACG CTCTAAGAAG |
| 1261 | AACAGCCTGG CCTTGTCCCT GACGGCCGAC CAGATGGTCA GTGCCTTGTT GGATGCTGAG |
| 1321 | CCCCCCATAC TCTATTCCGA GTATGATCCT ACCAGACCCT TCAGTGAAGC TTCGATGATG |
| 1381 | GGCTTACTGA CCAACCTGGC AGACAGGGAG CTGGTTCACA TGATCAACTG GGCGAAGAGG |
| 1441 | GTGCCAGGCT TTGTGGATTT GACCCTCCAT GATCAGGTCC ACCTTCTAGA ATGTGCCTGG |
| 1501 | CTAGAGATCC TGATGATTGG TCTCGTCTGG CGCTCCATGG AGCACCCAGT GAAGCTACTG |
| 1561 | TTTGCTCCTA ACTTGCTCTT GGACAGGAAC CAGGGAAAAT GTGTAGAGGG CATGGTGGAG |
| 1621 | ATCTTCGACA TGCTGCTGGC TACATCATCT CGGTTCCGCA TGATGAATCT GCAGGGAGAG |
| 1681 | GAGTTTGTGT GCCTCAAATC TATTATTTTG CTTAATTCTG GAGTGTACAC ATTTCTGTCC |
| 1741 | AGCACCCTGA AGTCTCTGGA AGAGAAGGAC CATATCCACC GAGTCCTGGA CAAGATCACA |
| 1801 | GACACTTTGA TCCACCTGAT GGCCAAGGCA GGCCTGACCC TGCAGCAGCA GCACCAGCGG |
| 1861 | CTGGCCCAGC TCCTCCTCAT CCTCTCCCAC ATCAGGCACA TGAGTAACAA AGGCATGGAG |
| 1921 | CATCTGTACA GCATGAAGTG CAAGAACGTG GTGCCCCTCT ATGACCTGCT GCTGGAGGCG |
| 1981 | GCGGACGCCC ACCGCCTACA TGCGCCCACT AGCCGTGGAG GGGCATCCGT GGAGGAGACG |
| 2041 | GACCAAAGCC ACTTGGCCAC TGCGGGCTCT ACTTCATCGC ATTCCTTGCA AAAGTATTAC |
| 2101 | ATCACGGGGG AGGCAGAGGG TTTCCCTGCC ACAGCTTGAT Gaagatctgag ctccctggcg |
| 2161 | gaattcggat cttattaaag cagaacttgt ttattgcagc ttataatggt tacaaataaa |
| 2221 | gcaatagcat cacaaatttc acaaataaag catttttttc actgcattct agttgtggtt |
| 2281 | tgtccaaact catcaatgta tcttatcatg tctggtcgac attaatgcta gcggcgcgcc |

Different ligands may differ in their affinity for alpha and beta isoforms of the estrogen receptor: 17-beta-estradiol binds equally well to both receptors; estrone, and raloxifene bind preferentially to the alpha receptor; and estriol, and genistein to the beta receptor. Subtype selective estrogen receptor modulators preferentially bind to either the α- or the β-subtype of the receptor. In addition, the different estrogen receptor combinations may respond differently to various ligands, which may translate into tissue selective agonistic and antagonistic effects. The ratio of α- to β-subtype concentration has been proposed to play a role in certain diseases. Both ERs are widely expressed in different tissue types, however there are some notable differences in their expression patterns. The ERα is found in endometrium, breast cancer cells, ovarian stroma cells, and the hypothalamus. In males, ERα protein is found in the epithelium of the efferent ducts. The expression of the ERβ protein has been documented in kidney, brain, bone, heart, lungs, intestinal mucosa, prostate, and endothelial cells. The ERs are regarded to be cytoplasmic receptors in their unliganded state, but visualization research has shown that a fraction of the ERs resides in the nucleus

The concept of selective estrogen receptor modulators is based on the ability to promote ER interactions with different proteins such as transcriptional coactivator or corepressors. Furthermore, the ratio of coactivator to corepressor protein varies in different tissues. As a consequence, the same ligand may be an agonist in some tissue (where coactivators predominate) while antagonistic in other tissues (where corepressors dominate). Tamoxifen, for example, is an antagonist in breast and is, therefore, used as a breast cancer treatment but an ER agonist in bone (thereby preventing osteoporosis) and a partial agonist in the endometrium (increasing the risk of uterine cancer)

### Estrogen Receptors and Cancer

Estrogen receptors are over-expressed in around 70% of breast cancer cases, referred to as “ER-positive”, and can be demonstrated in such tissues using immunohistochemistry. Two hypotheses have been proposed to explain why this causes tumorigenesis, and the available evidence suggests that both mechanisms contribute: (1) binding of estrogen to the ER stimulates proliferation of mammary cells, with the resulting increase in cell division and DNA replication, leading to mutation and (2) estrogen metabolism produces genotoxic waste.

The result of both processes is disruption of cell cycle, apoptosis and DNA repair, and, therefore, tumour formation. ERα is certainly associated with more differentiated tumours, while evidence that ERβ is involved is controversial. Different versions of the ESR1 gene have been identified (with single-nucleotide polymorphisms) and are associated with different risks of developing breast cancer.

Endocrine therapy for breast cancer involves selective estrogen receptor modulators (SERMS), such as tamoxifen, which behave as ER antagonists in breast tissue, or aromatase inhibitors, such as anastrozole. ER status is used to determine sensitivity of breast cancer lesions to tamoxifen and aromatase inhibitors. Another SERM, raloxifene, has been used as a preventive chemotherapy for women judged to have a high risk of developing breast cancer. Another chemotherapeutic anti-estrogen, ICI 182,780 (Faslodex), which acts as a complete antagonist, also promotes degradation of the estrogen receptor.

Estrogen and the ERs have also been implicated in breast cancer, ovarian cancer, colon cancer, prostate cancer, and endometrial cancer. Advanced colon cancer is associated with a loss of ERβ, the predominant ER in colon tissue, and colon cancer is treated with ERβ-specific agonists.

Phytoestrogens such as quercetin can modulate estrogen receptor's activities in such a way that it may prevent cancers including breasts, prostate, and colon all by promoting apoptosis. Quercetin selectively binds to the estrogen receptor beta (ERβ). Due to the ERβ being a ligand-activated transcription factor of which transcription is induced by estradiol, which allows the ERβ to bind to estrogen response elements located in the promoter region of the gene. This was tested in HeLa cells which were treated with a pure estrogen receptor antagonist which blocked both estradiol and quercetin from inducing the caspase-3 activation. ERβ is expressed in the human colon and activates a specific signal transduction pathway that controls apoptosis in the colon and works by being activated by estradiol and more recently found to possibly be activated by quercetin. Quercetin activates the ERβ along with the apoptotic cascade when caspase-3 is present by the phosphorylation of p38 kinase. In colon cancers and tumors ERβ and its pathway have been proven to be significantly decreased thus allowing the tumors to thrive.

### Sensorineural Hearing Loss (SNHL)

Sensorineural hearing loss is caused by death or dysfunction of several different cochlear cell types, including mechanosensory hair cells. Stem cell and gene therapies have been applied to the treatment of hearing loss in attempts to regenerate hair cells and restore hearing (Parker and Cotanche 2004; Raphael, Kim et al. 2007; Parker 2011). This patent expands this thesis to apply regenerative medicine to supporting cell biology with the goal of revering hearing loss. These heterogeneous cells are of interest because supporting cells act as hair cell progenitors in lower vertebrates, exhibit some capacity to differentiate into hair cells in mammals, and readily express exogenous transgenes. The overall goal of this patent is to use a novel genetic construct to regenerate auditory hair cells in a way that reflects the normal anatomy of the cochlea. To accomplish this goal, the patented transgene will allow for temporal and quantitative expression of the pro-hair cell gene Atoh1 in specific subpopulations of cochlear supporting cells.

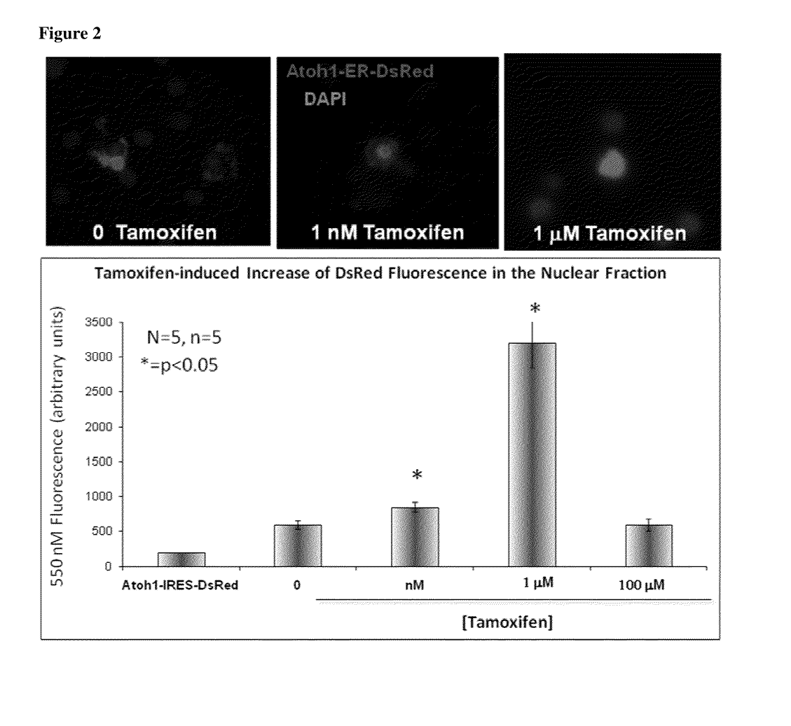

Each hair cell in the cochlea is surrounded by non-sensory supporting cells that provide trophic (Santos-Sacchi and Dallos 1983) and structural support for the hair cells (Raphael and Altschuler 2003) and ganglion neurons (Montcouquiol, Valat et al. 1998; Stankovic, R10 et al. 2004), and are essential in maintaining proper ionic concentrations in the organ of Corti through gap junction intercellular communication (Wangemann 2006; Zdebik, Wangemann et al. 2009). Supporting cells play a key role in hair cell regeneration. During development, hair and supporting cells develop from a common progenitor (Driver and Kelley 2009), and the appearance of a hair cell signals surrounding cells to develop into supporting cells through contact inhibition via the Notch signaling pathway (Kelley 2006). In animals such as birds that exhibit spontaneous hair cell regeneration after damage, the death of a hair cell triggers the adjacent supporting cell to either directly transdifferentiate into a regenerated hair cell, or to undergo mitosis to produce a new supporting cell and a regenerated hair cell (FIG. 2) (Stone and Cotanche 2007; Parker 2011).

Based on the ability of supporting cells to differentiate into hair cells, along with their shared developmental pathway, it has been postulated that supporting cells function as hair cell progenitors (Parker and Cotanche 2004). During development, the mammalian organ of Corti is as plastic as that of the chick, and several mitotic agents (i.e. retinoic acid (Kelley, Xu et al. 1993) and IGF (Malgrange, Rigo et al. 1999)) are capable of inducing supernumerary hair cells in the developing mammalian organ of Corti after the their normal genesis at approximately E13.5 ((Parker 2011)). Several studies have demonstrated that bypassing p27(Kip1)-dependent cell cycle inhibition in supporting cells can also result in hair cell regeneration in mammals (Lowenheim, Furness et al. 1999; Torchinsky, Messana et al. 1999; Minoda, Izumikawa et al. 2007). Therefore, similar to the chick cochlea, adult mammalian supporting cells maintain the ability to differentiate into hair cells once they are free to enter the cell cycle. However, several studies suggest that the ability for the cochlea to produce extra hair cells decreases as the organ ages (Kelley, Xu et al. 1993; Kwan, White et al. 2009).

### Polynucleotide Therapy

Nucleic acid molecules encoding therapeutic polypeptides of the invention can be delivered to cells (e.g., hair cells, stem cells). The nucleic acid molecules must be delivered to the cells of a subject in a form in which they can be taken up so that therapeutically effective levels of a reporter protein can be produced. Transducing viral (e.g., retroviral, adenoviral, and adeno-associated viral) vectors can be used, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding a therapeutic or reporter protein, variant, or a fragment thereof, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995). Retroviral vectors are particularly well developed and have been used in clinical settings (Rosenberg et al., N. Engl. J. Med 323:370, 1990; Anderson et al., U.S. Pat. No. 5,399,346). Most preferably, a viral vector is used to administer an expression vector of the invention to a target cell, tumor tissue, or systemically.

Non-viral approaches can also be employed for the introduction of a therapeutic to a cell (e.g., a tumor cell or neoplastic cell). For example, a nucleic acid molecule can be introduced into a cell by administering the nucleic acid molecule in the presence of lipofectin (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Expression of a therapeutic or reporter construct of the invention can be directed from any suitable promoter and regulated by any appropriate mammalian regulatory element. If desired, enhancers known to preferentially direct gene expression in specific cell types can be used to direct the expression of a nucleic acid (e.g., TAK1, GFAP, SRY, prox1). Nucleic acid sequences flanking the TAK1 gene are involved in the regulation of gene expression (GeneLoc location: GC06M091282, Start: 91,223,292 bp, End: 91,296,907 bp; e.g., 1, 2, 3, 4, 5, 6, 7, 8, 9, 10 kb upstream and/or 1, 2, 3, 4, 5, 6, 7, 8, 9, 10 kb downstream). An exemplary TAK1 promoter includes the sequence provided below (SEQ ID NO: 8):

| | |
|---|---|
| 1 | ATACTGACCA TCTTGGGGAC AGAATGATCT AGTTTGTGAG GACCATAATT GCTTTAAACT |
| 61 | CTCAGCGTGT TTTCTCATGT CCTGAAAATG GTGACAAAAA TATTCGATTT TCAAGGCGAC |
| 121 | TGTTGATGAT TCATTTACCA ATCTAAAGCA CTAGTGCCCC GCACTCGTAA TCCATAACTT |
| 181 | GAGATGAACA CACCAAATTA TAAATATCTT ATTCACCTAA TCCGTGGAAA GTTTGTAACA |
| 241 | CCAAAATATT AACTTCTGTT CTTGATCACT CAGCCTCCAT AGTTATTCAT GTTGCATGAT |
| 301 | CTGATTCGCT CTTTTAGAAT TTCTCATAGC GTCAGGTACT AGCTCAAAAT AACAGGAGCA |
| 361 | ACTGCTTCTT TCCATATCCC ACATTGAGGA AAACACATTT TGCAAGCCGT CTTAAAGAAA |
| 421 | CAGGTCAACT TAGTTTAGGG ATGAAACTTA AAAGTAAGCT TCGTCACAAA AAACACAGTT |
| 481 | CAACACCACC TCGCCCGGCT GCAGGCAGCT CAGAGGACAG GAAGTGAGGC CAAGACATAT |
| 541 | TTCACGCAGC ACAGACTAAA CCCCAGCTTT TATCTTTCCC GCTGGTCCTT AAGGGACCAA |
| 601 | CCGTACTGAT TTTCCGTCAT GTTCCGCGCC ACTTCCGTTC GCGCACTTTA ATTACGTCAG |
| 661 | GCGTCCTCTC TCGCGGTATC ATCCGGTTGC TGAGGCCCTG TAATAAAGGT CTCGCGAAAT |
| 721 | TTGTTCTAGA GGTCCAAGTT TGCTTCTTAG CTTACTCCAC CCCACCCCCA ACCTGTCCCT |
| 781 | CCTTTTCTTT CCAAGTCACA AAATTCTCCC CTCCCCTACC CCGGAGTTTA CGGCCCTCCT |
| 841 | CCTGTTTCCG ATTTCAGCCC GGAACCGGAA GTGTAGTGGG CGGGGCCCGT CGGCGGAAAA |
| 901 | CGCAGCGGAG CCAGAGCCGG ACACGGCTGT GGCCGCTGCC TCTACCCCCG CCACGGATCG |
| 961 | CCGGGTAGTA GGACTGCGCG GCTCCAGGCT GAGGGTCGGT CCGGAGGCGG GTGGGCGCGG |
| 1021 | GTCTCACCCG GATTGTCCGG GTGGCACCGT TCCCGGCCCC ACCGGGCGCC GCGAGGGATC |
| 1081 | ATGTCTACAG CCTCTG |

Alternatively, regulation can be mediated by cognate regulatory sequences or, if desired, by regulatory sequences derived from a heterologous source, including any of the promoters or regulatory elements described above. The invention provides for the expression of an expression vector comprising detectable reporters to indicate cellular localization.

### Reporter Expression

The invention further includes nucleic acid molecules that encode a reporter. Particularly useful in the methods of the invention are nucleic acid molecules encoding DsRed polypeptide, GFP polypeptide, poly histidine tag (His-tag), Human influenza hemagglutinin tag (HA-tag), flag tag (DYKDDDDK (SEQ ID NO: 9)) sequences, luciferase, or fragments thereof. The sequence of exemplary nucleic acid molecules are provided herein.

In general, detectable Atoh1-ER fusion polypeptides of the invention may be produced by transformation of a suitable host cell with all or part of an expression construct of the invention. Those skilled in the field of molecular biology will understand that any of a wide variety of expression systems may be used. The precise host cell used is not critical to the invention. A host cell is any cell (e.g., eukaryotic cell) that contains an expression vector.

A polypeptide of the invention may be produced in a eukaryotic host cell (e.g., a mammalian cells, e.g., NIH 3T3, HeLa, or preferably COS cells). Such cells are available from a wide range of sources (e.g., the American Type Culture Collection, Rockland, Md.; also, see, e.g., Ausubel et al., Current Protocol in Molecular Biology, New York: John Wiley and Sons, 1997). Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exist for the production of the polypeptides of the invention. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof. In one particular embodiment, the invention provides a lentiviral vector backbone comprising one or more polynucleotides encoding reporter constructs described herein. An expression vector is a nucleic acid construct, generated recombinantly or synthetically, bearing a series of specified nucleic acid elements that enable transcription of a particular gene in a host cell. Typically, gene expression is placed under the control of certain regulatory elements (e.g., 4OH-Tamoxifen, Tamoxifen, estrogen). Other regulatory elements include constitutive or inducible promoters, tissue-preferred regulatory elements, and enhancers (e.g., TAK1, GFAP, SRY, prox1). The invention provides for the expression of any of the detectable polypeptides described herein via an expression vector. The sequence of exemplary expression vectors are provided herein. In addition, the invention features host cells (e.g., mammalian, rodent, human cells) comprising a nucleic acid sequence that encodes any reporter described herein.

In another approach, an expression vector of the invention is expressed in a transgenic organism, such as a transgenic animal. By“transgenic” is meant any cell which includes a DNA sequence which is inserted by artifice into a cell and becomes part of the genome of the organism which develops from that cell, or part of a heritable extra chromosomal array. As used herein, transgenic organisms may be either transgenic vertebrates, such as domestic mammals (e.g., sheep, cow, goat, or horse), mice, or rats. In one embodiment, the reporter constructs of the invention are expressed in a transgenic animal, such as a rodent (e.g., a rat or mouse). In addition, cell lines from these mice may be established by methods standard in the art. Construction of transgenes can be accomplished using any suitable genetic engineering technique, such as those described in Ausubel et al. (Current Protocols in Molecular Biology, John Wiley & Sons, New York, 2000). Many techniques of transgene construction and of expression constructs for transfection or transformation in general are known and may be used for the disclosed constructs.

Animals suitable for transgenic experiments can be obtained from standard commercial sources such as Taconic (Germantown, N.Y.). Many strains are suitable, but Swiss Webster (Taconic) female mice are desirable for embryo retrieval and transfer. B6D2F (Taconic) males can be used for mating and vasectomized Swiss Webster studs can be used to stimulate pseudopregnancy. Vasectomized mice and rats are publicly available from the above-mentioned suppliers. However, one skilled in the art would also know how to make a transgenic mouse or rat (see, e.g., Helms et al., 2000).

### Formulation of Pharmaceutical Compositions

The administration of a compound or a combination of compounds of the invention may be by any suitable means that results in a concentration of the therapeutic that, combined with other components, is effective in treatment of sensorineural hearing loss or neoplasia. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Human dosage amounts can initially be determined by extrapolating from the amount of compound used in, for example, mice, as a skilled artisan recognizes it is routine in the art to modify the dosage for humans compared to animal models. In certain embodiments it is envisioned that the dosage may vary from between about 1 μg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 mg/Kg body weight. In other embodiments, it is envisaged that doses may be in the range of about 5 mg compound/Kg body to about 20 mg compound/Kg body. In other embodiments the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

The effective amount of a therapeutic agent (e.g., 4OHT) can be administered in a single dosage, two dosages or a plurality of dosages. Although it is to be understood that the dosage may be administered at any time, in one embodiment, the dosage is administered within 0.5, 1, 2, 3, 4, 5, 6, 7, 8, 9, 10, 11, or 12 hours after injury, or as soon as is feasible. In another embodiment, the dosage is administered to an injured mammal in one, two or a plurality of dosages; such dosages would be dependent on the severity of the injury. Where a plurality of dosages is administered, they may be delivered on a daily, weekly, or bi-weekly basis. The delivery of the dosages may be by means of catheter or syringe. Alternatively, the treatment can be administered during surgery to allow direct application to the auditory canal.

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target central nervous system injury or trauma by using carriers or chemical derivatives to deliver the therapeutic agent to a particular cell type (e.g., neuron). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

The compounds of the present invention can also be administered in combination with other active ingredients, such as, for example, adjuvants, protease inhibitors, or other compatible drugs or compounds where such combination is seen to be desirable or advantageous in achieving the desired effects of the methods described herein.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. Preferably, the composition may be administered locally, at or near the site of injury. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a nervous system injury or trauma, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

The present invention provides methods of treating cochlear injury, disease and/or disorders or symptoms thereof which comprise administering a therapeutically effective amount of a pharmaceutical composition comprising an agent described herein to a subject (e.g., a mammal such as a human). Thus, one embodiment is a method of treating a subject suffering from or susceptible to cochlear injury, disease or disorder or symptom thereof. The method includes the step of administering to the mammal a therapeutic amount of an agent herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for cochlear injury, disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of neoplasia.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with stroke or myocardial infarction in which the subject has been administered a therapeutic amount of a compound herein sufficient to treat the condition or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Solid Dosage Forms for Oral Use

Formulations for oral use include tablets containing active ingredient(s) (e.g., tamoxifen) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

The solid tablet compositions may include a coating adapted to protect the composition from unwanted chemical changes, (e.g., chemical degradation prior to the release of the active therapeutic substance). The coating may be applied on the solid dosage form in a similar manner as that described in Encyclopedia of Pharmaceutical Technology, supra.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

### Controlled Release Oral Dosage Forms

Controlled release compositions for oral use may, e.g., be constructed to release the active therapeutic by controlling the dissolution and/or the diffusion of the active substance. Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Methods of Treatment

In one embodiment, the present invention provides a method of treating sensorineural hearing loss or other type of hearing loss. Advantageously, the invention provides methods for increasing growth, proliferation, or survival of cochlear cells or hair cells, which may be used for treating hearing loss. Another aspect of the invention is the use of a compound of the invention in the manufacture of a medicament for increasing growth, proliferation, or survival of cochelar cells or hair cells in a subject. In addition to treating sensorineural hearing loss, the present invention may also be used in the treatment of neoplasia (e.g., colon, breast, and skin cancer). Atoh1 has been shown to act as a tumor suppressor. Without being bound to a particular theory, nuclear localization of Atoh1 is useful for its tumor suppressor activity. The methods involve administering to a subject in need of treatment, an effective amount of a therapeutic agent of the invention, for example, a vector expressing a polypeptide comprising an Atoh1-ER fusion protein. Preferably, such agents are administered via viral vector comprising a pharmaceutically acceptable carrier. Therapeutic agents (e.g., nucleic acids via viral or liposomal delivery, polypeptides) may be administered locally at the site of injury or systemically as to be effective, as is known to those skilled in the art. Additionally, an estrogen receptor ligand (e.g., 4OH-Tamoxifen, Tamoxifen, estrogen) is administered to the subject to achieve the therapeutic benefit of expressing the Atoh1-ER fusion protein. Preferably this method is employed to treat a subject suffering from or susceptible to hearing loss. Furthermore, the treatment methods of the invention can be used in combination with other available therapies for treating hearing loss Other embodiments include any of the methods herein wherein the subject is identified as in need of the indicated treatment. After a subject is diagnosed as having sensorineural hearing loss or other type of hearing loss injury, a method of treatment is selected. Preferably, the medicament is used for treatment or prevention in a subject of a disease, disorder or symptom set forth above. Thus, the invention provides methods for selecting a therapy for a subject, the method involving identifying a subject as having hearing loss (e.g., sensorineural hearing loss) or neoplasia (e.g., colon, breast, skin cancer), and administering to the subject a therapeutic composition of the invention.

### Methods for Evaluating Therapeutic Efficacy

In one approach, the efficacy of the treatment is evaluated by measuring, for example, the biological function of the treated organ (e.g., auditory function, hearing). Such methods are standard in the art and are described, for example, in the Textbook of Medical Physiology, Tenth edition, (Guyton et al., W.B. Saunders Co., 2000). In particular, a method of the present invention, increases the biological function of a tissue or organ by at least 5%, 10%, 20%, 40%, 50%, 60%, 70%, 80%, 90%, 100%, 150%, 200%, or even by as much as 300%, 400%, or 500%. Preferably, the tissue is coclear tissue and, preferably, the organ is the ear. Behavioral tests of recovery of function may also be used to evaluate treatment efficacy, including, for example, responses to auditory stimulation.

In another approach, the therapeutic efficacy of the methods of the invention is assayed by measuring an increase in cell number in the treated or transplanted tissue or organ as compared to a corresponding control tissue or organ (e.g., a tissue or organ that did not receive treatment). Preferably, the cell number in a tissue or organ is increased by at least 5%, 10%, 20%, 40%, 60%, 80%, 100%, 150%, or 200% relative to a corresponding tissue or organ. Methods for assaying cell proliferation are known to the skilled artisan and are described, for example, in Bonifacino et al., (Current Protocols in Cell Biology Loose-leaf, John Wiley and Sons, Inc., San Francisco, Calif.). For example, assays for cell proliferation may involve the measurement of DNA synthesis during cell replication. In one embodiment, DNA synthesis is detected using labeled DNA precursors, such as [³H]-Thymidine or 5-bromo-2*-deoxyuridine [BrdU], which are added to cells (or animals) and then the incorporation of these precursors into genomic DNA during the S phase of the cell cycle (replication) is detected (Ruefli-Brasse et al., Science 302(5650):1581-4, 2003; Gu et al., Science 302 (5644):445-9, 2003).

In another approach, efficacy is measured by detecting an increase in the number of viable cells in a tissue or organ relative to the number present in an untreated control tissue or organ, or the number present prior to treatment. Assays for measuring cell viability are known in the art, and are described, for example, by Crouch et al. (J. Immunol. Meth. 160, 81-8); Kangas et al. (Med. Biol. 62, 338-43, 1984); Lundin et al., (Meth. Enzymol. 133, 27-42, 1986); Petty et al. (Comparison of J. Biolum. Chemilum. 10, 29-34, .1995); and Cree et al. (AntiCancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett. 1: 611, 1991; Cory et al., Cancer Comm. 3, 207-12, 1991; Paull J. Heterocyclic Chem. 25, 911, 1988). Assays for cell viability are also available commercially. These assays include but are not limited to CELLTITER-GLO® Luminescent Cell Viability Assay (Promega), which uses luciferase technology to detect ATP and quantify the health or number of cells in culture, and the CellTiter-Glo® Luminescent Cell Viability Assay, which is a lactate dehyrodgenase (LDH) cytotoxicity assay (Promega).

Alternatively, or in addition, therapeutic efficacy is assessed by measuring a reduction in apoptosis. Apoptotic cells are characterized by characteristic morphological changes, including chromatin condensation, cell shrinkage and membrane blebbing, which can be clearly observed using light microscopy. The biochemical features of apoptosis include DNA fragmentation, protein cleavage at specific locations, increased mitochondrial membrane permeability, and the appearance of phosphatidylserine on the cell membrane surface. Assays for apoptosis are known in the art. Exemplary assays include TUNEL (Terminal deoxynucleotidyl Transferase Biotin-dUTP Nick End Labeling) assays, caspase activity (specifically caspase-3) assays, and assays for fas-ligand and annexin V. Commercially available products for detecting apoptosis include, for example, Apo-ONE® Homogeneous Caspase-3/7 Assay, FragEL TUNEL kit (ONCOGENE RESEARCH PRODUCTS, San Diego, Calif.), the ApoBrdU DNA Fragmentation Assay (BIOVISION, Mountain View, Calif.), and the Quick Apoptotic DNA Ladder Detection Kit (BIOVISION, Mountain View, Calif.).

### Kits or Pharmaceutical Systems

The present compositions may be assembled into kits or pharmaceutical systems for use in the growth, proliferation, or survival of cochlear cells and hair cells. The compositions of the kits or pharmaceutical systems may be used for treating sensorineural hearing loss or other trauma involving hearing loss. In other embodiments, the compositions of the kits or pharmaceutical systems may be used for neoplasia (e.g., colon, breast, skin). Kits or pharmaceutical systems according to the invention comprise a carrier means, such as a box, carton, tube or the like, having in close confinement therein one or more container means, such as vials, tubes, ampoules, bottles and the like. The kits or pharmaceutical systems of the invention may also comprise associated instructions for using the agents of the invention. Kits of the invention include at least a polynucleotide encoding an Atoh1 fused to the ligand binding domain of an Estrogen receptor. In particular embodiments, the Atoh1-ER fusion protein may further comprise a reporter (e.g., DsRed). In some embodiments, the kit may include one or more of an estrogen ligand, including 4-hydroxy tamoxifen, tamoxifen, and estrogen. The kit may include instructions for administering the polynucleotide encoding an Atoh1-ER fusion protein in combination with one or more agents that bind the ligand binding domain of the estrogen receptor. Methods for measuring the efficacy of agents with 4-sulfatase activity are known in the art and are described herein.

### EXAMPLES

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1

### Tamoxifen-Dependent Nuclear Translocalization of the Atoh1-ER-DsRed Fusion Protein

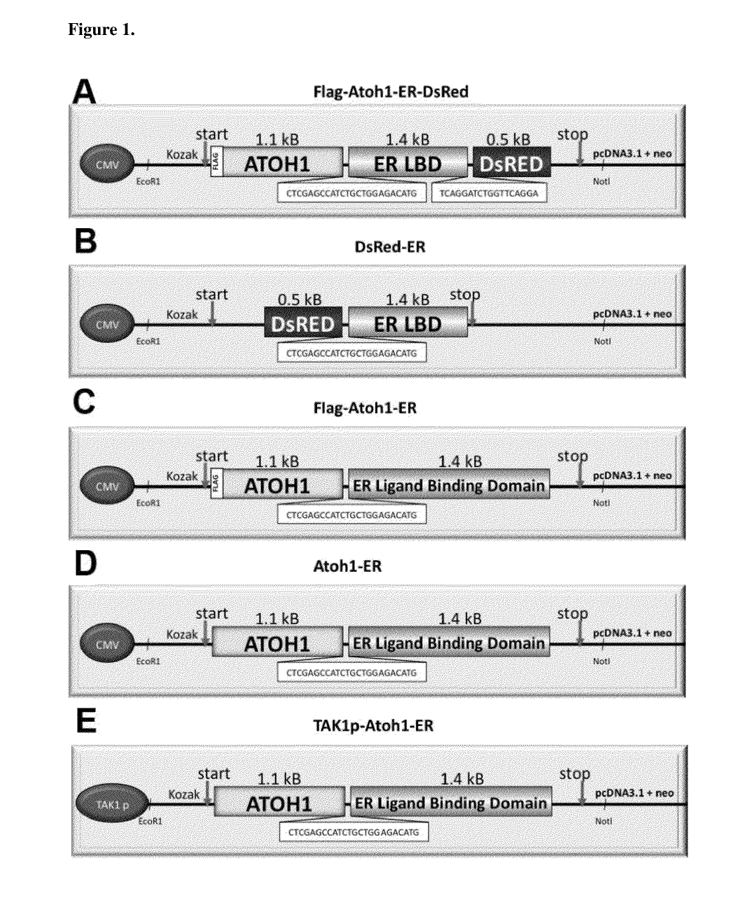

Constructs were generated to express inducible Atoh1-ER fusion proteins (FIG. 1). HEK cells transfected with the Atoh1-ER-DsRed construct constitutively express a Atoh1-ER-DsRed fusion protein that can be easily detected by the fluorescence of the DsRed moiety. In the Atoh1-ER-DsRed construct, Atoh1 has been fused to estrogen receptor ligand binding domain variant that limits endogenous 17b-estradiol binding at physiological concentrations (Danielian et al., 1998; Danielian et al., 1993). As can be seen using fluorescence microscopy, in the absence of 4OHT, the Atoh1-ER-DsRed fusion protein remains sequestered within the cytosol of transfected cells (FIG. 2). To quantify the nuclear translocation of the Atoh1-ER-DsRed fusion protein, HEK cells were transfected with this construct, incubated in graded doses of 4OHT for 72 hrs, subjected to nuclear fractionation, and their isolated nuclei were pooled and mounted on coverslips for analysis of DsRed fluorescence.

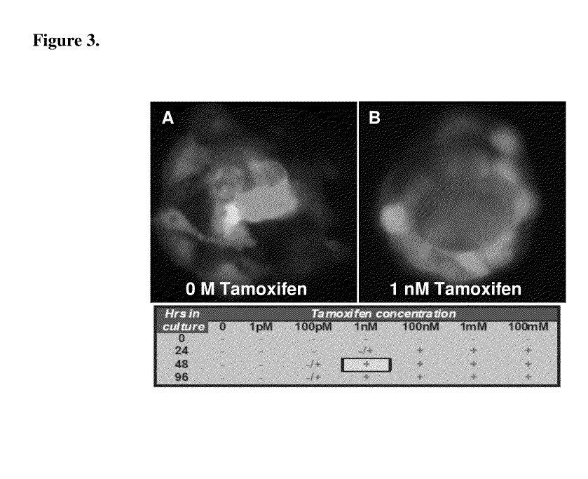

The results showed that control groups that were transfected with the CMV-Atoh1-Ires-DsRed exhibited a low level of background 568 nM fluorescence (194.7+/−0.27 mean arbitrary units [au]). Groups of cells transfected with CMV-Atoh1-ER-DsRed and not exposed to tamoxifen exhibited an insignificant increase in 588 nM fluorescence (392.0+/−68.35 au; p=0.16), which suggested that there is a low level of nuclear translocalization of the Atoh1-ER-DsRed fusion protein in the absence of tamoxifen. Incubating the cultures in 1 nM (848.0+/−29.33 au; p<0.05) and 1 mM (3,200.9+/−156.95 au; p<0.05) 4OHT for 72 hrs resulted in a significant increase in 568 nM fluorescence in the nuclear fraction, which demonstrates a 4OHT dose dependent-increase translocation of Atoh1-ER-DsRed to the nucleus. Removal of 4OHT from the culture media results in a complete cytoplasmic localization of the fusion protein within 2 weeks (data not shown). However, incubation with 100 mM 4OHT for three days was toxic to the cells and the remaining nuclei contained 568 nM fluorescence that was statistically equivalent to groups receiving no 4OHT (597.8+/−40/46; p=0.07). The minimum effective dose was empirically determined to be 1 nM 4OHT for 2-7 days (FIG. 3).

### Example 2

### Tamoxifen-Dependent Binding of the Atoh1-ER-DsRed Fusion Protein to the 5′ Atoh1 Enhancer/Promoter

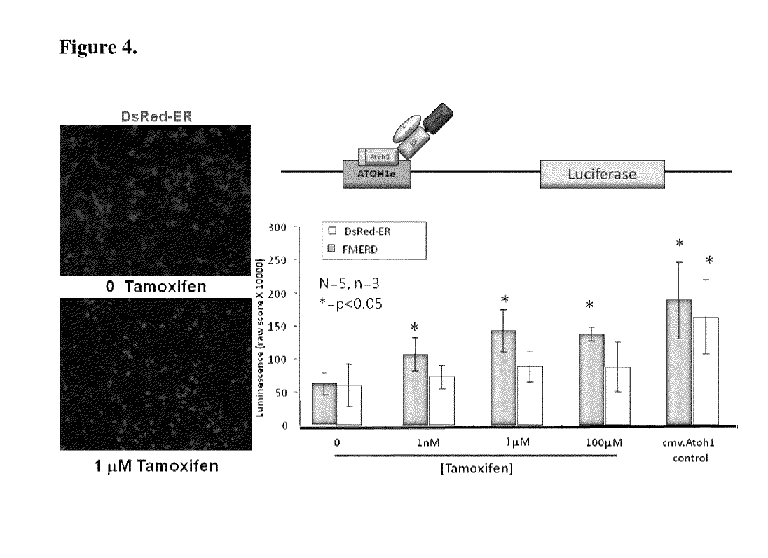

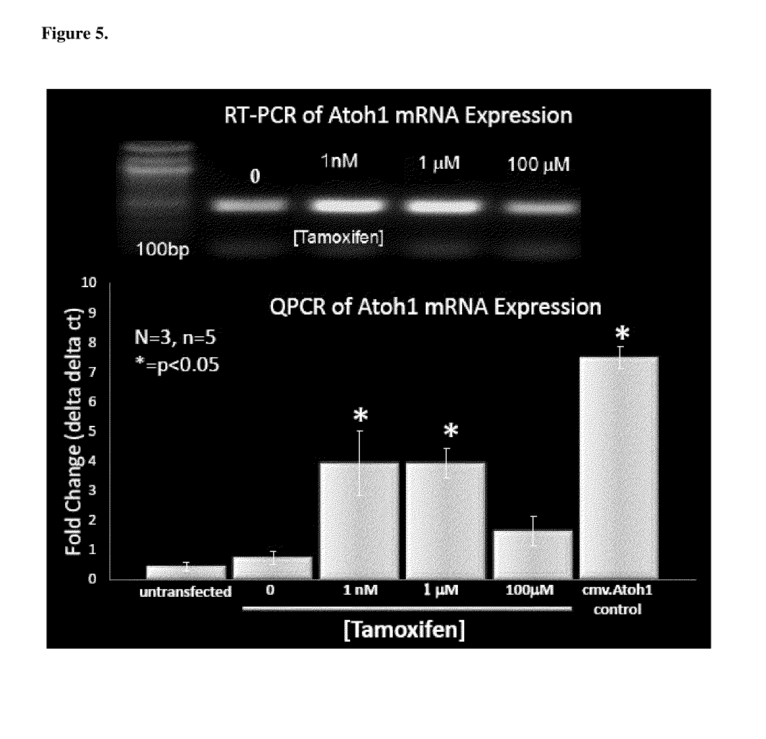

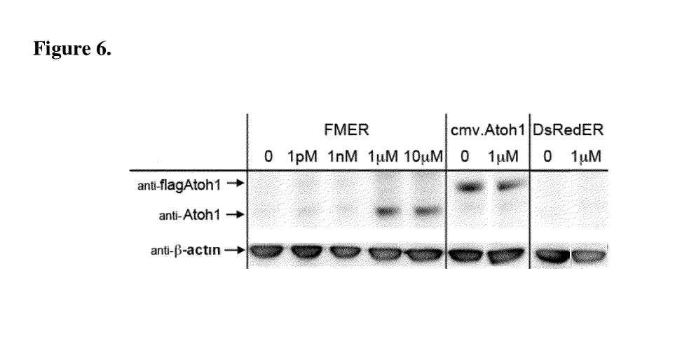

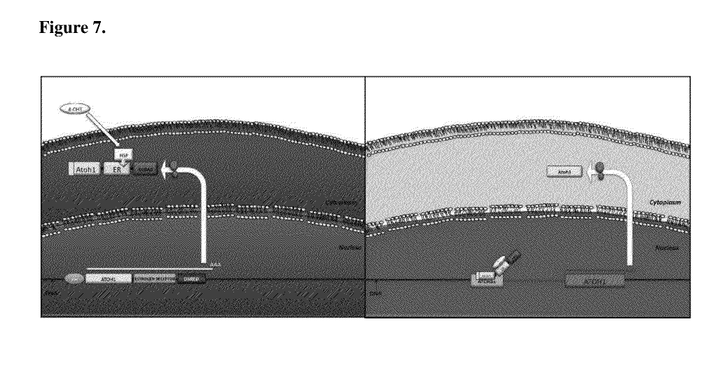

Because Atoh1 acts as a feed-forward autoregulatory transcription factor, whereby it acts to positively regulate itself, the effects of 4OHT on Atoh1 gene expression were examined. Increasing concentrations of 4OHT induced a dose-dependent increase in binding to the enhancer/promoter region of the Atoh1 gene (Helms et al., 2000) measured by luciferase binding assay (FIG. 4). Additionally, both RT-PCR and qPCR indicated that 4OHT upregulates the expression of Atoh1 in a dose-dependent manner (FIG. 5). Western blot analysis determined that 4OHT increased the concentration of cytosolic Atoh1 protein in a dose-dependent manner (FIG. 6). Without being bound to a particular theory, 4-OHT competes with HSP90 and allows the Atoh1-ER-DsRed fusion protein to translocate to the nucleus where it binds to the endogenous Atoh1 enhancer/promoter region and expresses endogenous Atoh1 in a feed-forward mechanism (FIG. 7).

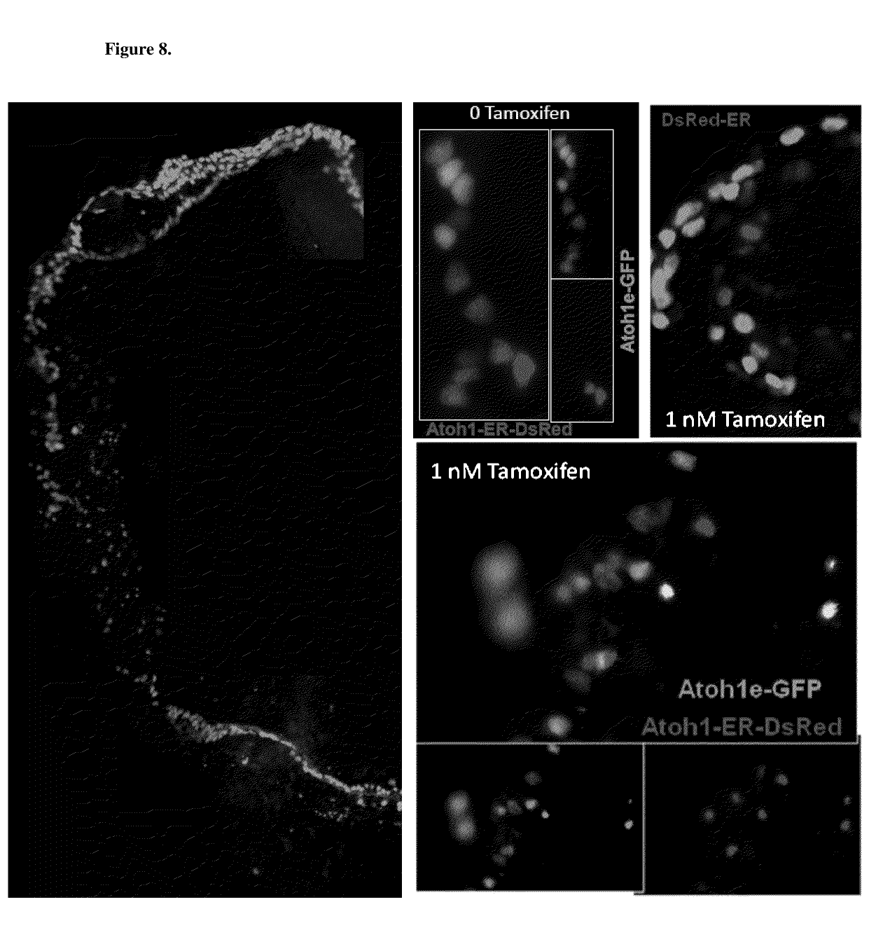

It was determined whether 4OHT induced Atoh1 expression was sufficient for activation of downstream signaling pathways. To test this, the expression of myosin 7a, which is a downstream target of Atoh1 signaling, was measured in HEI OC1 cells transfected with the Atoh1-ER-DsRed construct. Proliferating HEI OC1 cells were transfected with the Atoh1-ER-DsRed construct, then these cells were cultured as floating aggregates in differentiating conditions (39° C.) in either the presence or absence of 1 mM 4OHT for three days, before fixing them and measuring immunolabeling to myosin 7a (FIG. 8). There was no significant difference in the numbers DsRed positive cells (p=0.07) between the groups receiving 4OHT or not, which suggests similar transfection efficiencies between these two groups. However, the transfected cells that were incubated with 4OHT exhibited a significant increase (p<0.001) in myosin 7a labeling (79%; +/−5%) when compared to transfected cells incubated in vehicle alone (26%; +/−6%).

The construct was electroporated into neonatal organs of Corti isolated from transgenic mouse pups that express GFP under control of the 5′ Atoh1 enhancer/promoter. These results indicate that the Atoh1-ER-DsRed fusion protein upregulates Atoh1 expression in organ of Corti explants in culture. Taken together, these data indicated that the Atoh1-ER-DsRed fusion protein may be used to both upregulate and down regulate Atoh1 expression by administering 4OHT in a dose-dependent manner.

A genetic construct where Atoh1 can be modulated with 4OHT may be directly transfected into tissue for in vitro or in vivo analysis of regulated Atoh1 expression. An advantage over constitutively expressing systems is the ability to control the temporal and quantitative expression of Atoh1. Atoh1-ER-DsRed construct can be packaged in viral particles and infected into sound damaged cochleas. Using a reporter construct, temporal and quantitative expression of Atoh1 can be analyzed in a translational model without requiring breeding multiple generations of transgenic organisms. Cell specific expression of Atoh1 by placing the Atoh1-ER-DsRed construct under control of cell-specific promoters such as Glial fibrillary acidic protein (GFAP; R10 et al., 2002), SRY (sex determining region Y)-box 2 (Sox2; Hume et al., 2007), Prospero homeobox protein 1 (prox1; Bermingham-McDonogh et al., 2007), and Transforming Growth Factor β-activated Kinase 1 (TAK1; Parker et al. 2011), which is a specific marker for adult supporting cells in the cochlea, can be used in translational delivery systems for expressed Atoh1 in a temporal, quantitative, and cell-specific locations within the organ of Corti.

Results reported herein were obtained using the following methods and materials unless indicated otherwise.

### Generation of the Atoh1-ER-DsRed and Control Constructs.

Constructs designed for the experiments are shown in FIGS. 1A-1E, 10, and 11. Constitutively expressing positive control constructs were generated comprising an Atoh1 sequence that was modified by PCR cloning to include two consecutive flag tag sequences (GATTACAAGGATGACGATGACAAG (SEQ ID NO: 10)) preceding the start codon. For the construct encoding an inducible Atoh1-ER-DsRed transgene, PCR cloning primers were designed so that 1) an EcoRI site was placed on the 5′ end and a Kozac sequence (CACC) was engineered upstream of the Atoh1 start codon; 2) the Atoh1 stop codon (TAG) was deleted; 3) this same flag tagged Atoh1 sequence from above was linked to an ER LBD sequence by the sequence CTCGAGCCATCTGCTGGAGACATG (SEQ ID NO: 1) encoding a polypeptide linker; 4) the ER LBD stop codon (TAG) was deleted; 5) the ER LBD sequence was linked to a DsRed sequence by the sequence TCAGGATCTGGTTCAGGA (SEQ ID NO: 2) encoding a polypeptide linker; and 6) a Not I site was included on the 3′ end. The linker sequences were designed to translate into multiple proline sequences which provide an increased degree of freedom for the subunits of the fusion protein. The insert for the ER construct was amplified using a 2-step PCR from template DNA (provided by A. McMahon, Harvard Medical School) that has been mutated to limit endogenous 17b-estradiol binding at physiological concentrations (Danielian, P. S., et al., Curr Biol, 1998. 8(24): p. 1323-6; Danielian et al., Mol Endocrinol, 1993. 7(2): p. 232-40). DsRed DNA was obtained from a commercial vector (Clonetech). Finally, to make the negative control (DsRed-ER) construct, PCR cloning primers were designed so that 1) an EcoRI site was placed on the 5′ end and a Kozac sequence (CACC) was placed upstream of the DsRed start codon; 2) the stop codon (TAG) for DsRed was deleted; 3) DsRed was linked to an ER LBD sequence by the sequence TCAGGATCTGGTTCAGGATCCATG (SEQ ID NO: 3) encoding a polypeptide linker; and 4) a Not1 site was cloned onto the 3′ end.

Constructs were subcloned into the multiple cloning site of the pcDNA3.1(+) vector which employs a cmv promoter to drive gene expression. For cochlear specific expression, TAK1 promoter was used in place of the CMV promoter. To accomplish this, inserts for Atoh1-ER-DsRed and DsRed-ER constructs were amplified using a 2-step PCR from template DNA. AccuPrime™ Pfx SuperMix (Invitrogen) was used for the PCR amplification. The PCR products were gel purified, digested with EcoRI and Not I, and purified with PureLink PCR Purification Kit (Invitrogen) as inserts. Next, 10 μg of pcDNA3.1(+) was digested with EcoRI and Not I for 2 hrs at 37° C. Calf intestinal alkaline phosphatase (10) (Invitrogen) was added to the digestion solution and incubated at 37° C. for 10 minutes. The digest was phenol extracted, ethanol precipitated, washed with 80% ethanol and resuspended in sterile water. Ligations were performed using T4 DNA Ligase (Invitrogen), using fusion fragments as insert and pcDNA3.1(+) as vector at a ratio of 3:1 (insert:vector). The ligations were transformed into TOP10 cells and equal volumes were plated on LB/Amp (100 m/ml) plates. Sixteen colonies for each desired construct were picked for colony PCR with vector primers T7 and BGH reverse. Positive colonies were mini prepped with PureLink HQ Mini Plasmid DNA Purification Kit (Invitrogen), and verified by restriction digest with EcoRI and Not I. Positive clones were sequenced with vector primers T7 and BGH reverse and gene specific Atoh1-ER-DsRed (TTGTGTGCCTCAAATCCATC (SEQ ID NO: 11), CCTTACAAACCTACTACATACC (SEQ ID NO: 12)) or DsRed-ER(CCCGTAATGCAGAAGAAGAC (SEQ ID NO: 13), GGTCAGTGCCTTGTTGGATG (SEQ ID NO: 14)) sequencing primers to verify the cloning junctions and orientation. Glycerol stocks were then prepared from positive clones and stored at −80° C. for further use.

Some expression constructs where Atoh1 was directly fused to the ER and/or DsRed moieties or where ER and/or DsRed were fused upstream of Atoh1 were less effective in up-regulating endogenous Atoh1. Adding a linking sequence between the Atoh1 ER and DsRed fusion constructs also allowed for greater Atoh1 expression. Without being bound to a particular theory, linking sequences decrease the steric hindrance and, therefore, increase the degrees of freedom between these moieties.

### Generation and Electroporation of Cochlear Spheres.

Cochlear derived progenitor cells were generated and floating aggregates (cochlear spheres) propagated as previously described (Oshima et al., Journal of the Association for Research in Otolaryngology, 2007. 8(1): 18-31) with the following modifications. Cochleas were isolated from litters of P0-P3 ROSA26-GFP mice, the organs of Corti were dissected, pooled, trypsinized, triturated, and centrifuged. The pellet was re-suspended in SFM, filtered through a 70 μM cell strainer, and cultured for 5 days in this same media supplemented with growth factors (10 ng/ml of FGF, IGF, EGF, Heparin sulfate). Floating aggregates were collected, centrifuged, triturated using a 100 μl pipette, re-suspended in 300 μl Optimem, and electroporated (8 pulses; 25 V; duration, 50 ms; interval, 100 ms with 2 mg/ml DNA in water, and incubated in 3:1 Fugene 6 overnight) using 50 μg of plasmid DNA. Spheres derived from the experimental (Atoh1-ER-DsRed) and control groups (cmv.flagAtoh1 and DsRed-ER) were expanded by culturing on 6-well plates for an additional 5 days in the same media at 37° C., and then incubated in graded doses of 1 nM 4-hydroxy tamoxifen sulfate (4OHT) for 72 hrs (N=10 for each dosage). Finally, spheres from each of these groups were centrifuged, adhered to glass coverslips by incubation for 2-4 hours at 37° C. on glass coverslips coated with 1:1 poly-lysine/polyornithine, fixed in 4% paraformaldehyde for 20 minutes, washed three times in PBS, and stored at 4° C. for later analysis.

### Nuclear Translocalization Assay.

HEK cells were cultured until 50% confluent in 6-well culture dishes (type) then subjected to transfection using 3:1 target DNA to Fugene 6 Transfection Reagent (ROCHE). Cells were incubated for 24 hours, and then incubated with graded doses of 4OHT for 5-7 days. Cells were then processed for cytosolic and nuclear fractionation (BioVision). The isolated nuclear fraction collected from each of 5 sample wells per condition was mounted to a coverslip and average pixel density from 5 regions of interest (206.5×165.2 pixel at 20× magnification) was measured with a Cy3 (550 nM) filter on a Zeiss epifluorescent microscope using MetaMorph software.

### Luciferase Assay.

The Atoh1 5′ enhancer/promoter region (Helms, Abney et al. 2000) was cloned into the MCS of the pGL3-Promoter Luciferase Reporter Vector (Promega), and was stably expressed on a HEK cell line using selection to ampicillin. These cells were grown until 80% confluent on 6-well plates, and then transiently transfected with either the cmv.Atoh1 control vector, the cmv.Atoh1-ER-DsRed construct, or a cmv.DsRed-ER negative control construct using 3:1 target DNA to Fugene 6 Transfection Reagent. All cells were also co-transfected with Renilla transfection controls. Cells were incubated for 72 hrs in increasing doses of 4OHT, then washed on PBS, lysed and subjected to Dual-Luciferase Reporter Assay (Promega). Firefly luciferase activity was measured in a manual TD-20/20 Luminometer (Turner Designs).

### RNA Analysis.

HEK cells were grown on 6-well plates until 80% confluent, then were transiently transfected with either the cmv.Atoh1-ER-DsRed or cmv.Atoh1 construct as described above and incubated for 72 hrs with different doses of 4OHT. Next, total RNA was extracted from the cells by adding 1 ml Trizol reagent (Invitrogen) to each well for 5 min, cells were scraped into a 1.5 ml tube (1 tube/well), incubated with 200 ml chloroform (in hood) for 2 min centrifuged for 20 min at 12,000 g at 4° C., supernatant was collected in a new 1.5 ml tube, incubated with 1:1 equivalent volume of 2-propanol equal volume to supernatant, and centrifuged through the RNeasy mini kit columns at 8000 g for 15 sec. RNA was eluted from the column by adding 700 ml RW1, centrifuging the column at 8000 g 15 s, adding 2×500 ml washes of RPE2 and re-centrifuging at 8000 g 15 s, adding one spin to dry membrane (10,000 g, 1 min), and eluting the RNA by adding 45 ml RNAse free water into new 1.5 ml tube and centrifuging a final time at 8000 g for 15 s.

For the reverse transcriptase polymerase chair reactions (RT-PCR), 45 μL of template RNA was added to a PCR tube and mixed with 20 μL 5× first strand buffer, 11 μL 50 mM MgCl2, 54, dNTP (10 mM), 5 μL random primers (Invitrogen), 1.1 μL each of forward (aga tct aca tca acg ctc tgt c) and reverse primers (act ggc ctc atc aga gtc act g) designed to amplify 449 base pair segment of the Atoh1 cDNA, 13 μL dH2O for a total reaction volume of 100 μL. The hexamers were incubated at 25° C. for 10 min, the RT reaction consisted of 37° C. for 60 min, and RT incubation was 95° C. for 5 min, held at 4° C., and stored on ice until run on 1% agarose gels for analysis.

For quantitative PCR (qPCR) analysis, 300 μL of qPCR Master Mix (Invitrogen) was added to a PCR tube with 300 μL dH2O, which was then divided into 5 tubes (120 μL each). Six μL of template cDNA was added to each tube, which were then divided into two wells in which 3 μL of probe was added in a 96-well plate (TempPlateIII PCR plate USA Scientific) (18 s standard in column 1, Atoh1 in column 4), mixed by pipeting up and down, split by adding 20 μL from column 1 to column 2 and 3 and then adding 20 μL from column 4 to column 5 and 6. The 96-well plate was covered with optically clear film, and bubbles on the bottom of the wells were shaken away. Quantitative PCR was performed and the amount of RNA was determined Delta delta Ct measurements were calculated for each treatment group, and then were normalized to fold change from groups incubated in the absence of tamoxifen. Mean fold change for each experimental condition were averaged and subjected to students t-test for significance testing.

### Western Blot Analysis.

HEK cells were grown to 80% confluence in 10 mm culture plates (types), transiently transfected with the Atoh1-ER-DsRed construct using 3:1 target DNA to Fugene 6 Transfection Reagent, and incubated with graded doses of 4OHT for 72 hours at 37° C. Control samples were similarly transfected with either DsRed-ER (negative control) or a positive control vector (cmv.flagAtoh1). Cells were lysed, the whole cell protein was collected and processed for Western blot analysis using either anti-Atoh1polyclonal antibody (Developmental Studies Hybridoma Bank) or a polyclonal anti-b-actin antibody (Sigma).

### Organ of Corti Dissection, Culture and Electroporation.

A detailed protocol for this procedure has been described (Parker et al., Journal of Visualized Experiments, 2010(36). Briefly, the organs of Corti were dissected from P0-P3 mice pups that express a nuclear targeted GFP under control of the Atoh1 enhancer/promoter (gift from Jane Johnson) Helms et al., Development, 2000. 127(6): p. 1185-96, cultured overnight on 1:1 poly-lysine/ornithine glass coverslips in 10% serum, and then electroporated with 2 mg/ml target DNA. Organs of Corti were returned to the incubator and incubated in the presence or absence of 4OHT for 48 hours. Next, 2 mL of serumed media was added to the wells and the organs were incubated at 37° C. for 5 days, then fixed in 4% paraformaldehyde for 20 min, washed three times in HBSS, and processed for immunofluorescent labeling to myosin 7a.

The sequences of pcDNA3.1-Flag-Atoh1-ER-dsRed and Flag-Atoh1-ER-Fusion are provided below.

The nucleic acid sequence of a vector encoding Flag-Atoh1-ER-DsRed (pcDNA3.1Flag-Atoh1-ER-dsRed (SEQ ID NO: 15)) is depicted below:

| | |
|---|---|
| 1 | maghlasdfa fspppggggd gpggpepgwv dprtwlsfqg ppggpgigpg vgpgsevwgi |
| 1 | gacggatcgg gagatctccc gatcccctat ggtgcactct cagtacaatc tgctctgatg |
| 61 | ccgcatagtt aagccagtat ctgctccctg cttgtgtgtt ggaggtcgct gagtagtgcg |
| 121 | cgagcaaaat ttaagctaca acaaggcaag gcttgaccga caattgcatg aagaatctgc |
| 181 | ttagggttag gcgttttgcg ctgcttcgcg atgtacgggc cagatatacg cgttgacatt |
| 241 | gattattgac tagttattaa tagtaatcaa ttacggggtc attagttcat agcccatata |
| 301 | tggagttccg cgttacataa cttacggtaa atggcccgcc tggctgaccg cccaacgacc |
| 361 | cccgcccatt gacgtcaata atgacgtatg ttcccatagt aacgccaata gggactttcc |
| 421 | attgacgtca atgggtggag tatttacggt aaactgccca cttggcagta catcaagtgt |
| 481 | atcatatgcc aagtacgccc cctattgacg tcaatgacgg taaatggccc gcctggcatt |
| 541 | atgcccagta catgacctta tgggactttc ctacttggca gtacatctac gtattagtca |
| 601 | tcgctattac catggtgatg cggttttggc agtacatcaa tgggcgtgga tagcggtttg |
| 661 | actcacgggg atttccaagt ctccacccca ttgacgtcaa tgggagtttg ttttggcacc |
| 721 | aaaatcaacg ggactttcca aaatgtcgta acaactccgc cccattgacg caaatgggcg |
| 781 | gtaggcgtgt acggtgggag gtctatataa gcagagctct ctggctaact agagaaccca |
| 841 | ctgcttactg gcttatcgaa attaatacga ctcactatag ggagacccaa gctggctagc |
| 901 | gtttaaactt aagcttggta ccgagctcgg atccactagt ccagtgtggt ggaattcgcc |
| 961 | accatggact acaaagacga tgatgataaa gattacaaag atgacgatga caaggggtcc |
| 1021 | cgcctgctgc atgcagaaga gtgggctgag gtaaaagagt tgggggacca ccatcgccat |
| 1081 | ccccagccgc accacgtccc gccgctgacg ccacagccac ctgctaccct gcaggcgaga |
| 1141 | gaccttcccg tctacccggc agaactgtcc ctcctggata gcaccgaccc acgcgcctgg |
| 1201 | ctgactccca ctttgcaggg cctctgcacg gcacgcgccg cccagtatct gctgcattct |
| 1261 | cccgagctgg gtgcctccga ggccgcggcg ccccgggacg aggctgacag ccagggtgag |
| 1321 | ctggtaagga gaagcggctg tggcggcctc agcaagagcc ccgggcccgt caaagtacgg |
| 1381 | gaacagctgt gcaagctgaa gggtggggtt gtagtggacg agcttggctg cagccgccag |
| 1441 | cgagcccctt ccagcaaaca ggtgaatggg gtacagaagc aaaggaggct ggcagcaaac |
| 1501 | gcaagggaac ggcgcaggat gcacgggctg aaccacgcct tcgaccagct gcgcaacgtt |
| 1561 | atcccgtcct tcaacaacga caagaagctg tccaaatatg agaccctaca gatggcccag |
| 1621 | atctacatca acgctctgtc ggagttgctg cagactccca atgtcggaga gcaaccgccg |
| 1681 | ccgcccacag cttcctgcaa aaatgaccac catcaccttc gcaccgcctc ctcctatgaa |
| 1741 | ggaggtgcgg gcgcctctgc ggtagctggg gctcagccag ccccgggagg gggcccgaga |
| 1801 | cctaccccgc ccgggccttg ccggactcgc ttctcaggcc cagcttcctc tgggggttac |
| 1861 | tcggtgcagc tggacgcttt gcacttccca gccttcgagg acagggccct aacagcgatg |
| 1921 | atggcacaga aggacctgtc gccttcgctg cccgggggca tcctgcagcc tgtacaggag |
| 1981 | gacaacagca aaacatctcc cagatcccac agaagtgacg gagagttttc cccccactct |
| 2041 | cattacagtg actctgatga ggccagtctc gagccatctg ctggagacat gagggctgcc |
| 2101 | aacctttggc caagccctct tgtgattaag cacactaaga agaatagccc tgccttgtcc |
| 2161 | ttgacagctg accagatggt cagtgccttg ttggatgctg aaccgcccat gatctattct |
| 2221 | gaatatgatc cttctagacc cttcagtgaa gcctcaatga tgggcttatt gaccaaccta |
| 2281 | gcagataggg agctggttca tatgatcaac tgggcaaaga gagtgccagg ctttggggac |
| 2341 | ttgaatctcc atgatcaggt ccaccttctc gagtgtgcct ggctggagat tctgatgatt |
| 2401 | ggtctcgtct ggcgctccat ggaacacccg gggaagctcc tgtttgctcc taacttgctc |
| 2461 | ctggacagga atcaaggtaa atgtgtggaa ggcatggtgg agatctttga catgttgctt |
| 2521 | gctacgtcaa gtcggttccg catgatgaac ctgcagggtg aagagtttgt gtgcctcaaa |
| 2581 | tccatcattt tgcttaattc cggagtgtac acgtttctgt ccagcacctt gaagtctctg |
| 2641 | gaagagaagg accacatcca ccgtgtcctg gacaagatca cagacacttt gatccacctg |
| 2701 | atggccaaag ctggcctgac tctgcagcag cagcatcgcc gcctagctca gctccttctc |
| 2761 | attctttccc atatccggca tatgagtaac aaacgcatgg agcatctcta caacatgaaa |
| 2821 | tgcaagaacg tggtacccct ctatgacctg ctcctggaga tgttggatgc ccaccgcctt |
| 2881 | catgccccag ccagtcgcat gggagtgccc ccagaggagc ccagccagac ccagctggcc |
| 2941 | accaccagct ccacttcagc acattcctta caaacctact acataccccc ggaagcagag |
| 3001 | ggcttcccca acacgatctc aggatctggt tcaggagcca caaccatggc ctcctccgag |
| 3061 | gacgtcatca aggagttcat gcgcttcaag gtgcgcatgg agggctccgt gaacggccac |
| 3121 | gagttcgaga tcgagggcga gggcgagggc cgcccctacg agggcaccca gaccgccaag |
| 3181 | ctgaaggtga ccaagggcgg ccccctgccc ttcgcctggg acatcctgtc cccccagttc |
| 3241 | cagtacggct ccaaggtgta cgtgaagcac cccgccgaca tccccgacta caagaagctg |
| 3301 | tccttccccg agggcttcaa gtgggagcgc gtgatgaact tcgaggacgg cggcgtggtg |
| 3361 | accgtgaccc aggactcctc cctgcaggac ggctccttca tctacaaggt gaagttcatc |
| 3421 | ggcgtgaact tcccctccga cggccccgta atgcagaaga agactatggg ctgggaggcc |
| 3481 | tccaccgagc gcctgtaccc ccgcgacggc gtgctgaagg gcgagatcca caaggccctg |
| 3541 | aagctgaagg acggcggcca ctacctggtg gagttcaagt ctatctatat ggccaagaag |
| 3601 | cccgtgcagc tgcccggcta ctactacgtg gactccaagc tggacatcac ctcccacaac |
| 3661 | gaggactaca ccatcgtgga gcagtacgag cgcgccgagg gccgccacca cctgttcctg |
| 3721 | taggcggccg ctcgagtcta gagggcccgt ttaaacccgc tgatcagcct cgactgtgcc |
| 3781 | ttctagttgc cagccatctg ttgtttgccc ctcccccgtg ccttccttga ccctggaagg |
| 3841 | tgccactccc actgtccttt cctaataaaa tgaggaaatt gcatcgcatt gtctgagtag |
| 3901 | gtgtcattct attctggggg gtggggtggg gcaggacagc aagggggagg attgggaaga |
| 3961 | caatagcagg catgctgggg atgcggtggg ctctatggct tctgaggcgg aaagaaccag |
| 4021 | ctggggctct agggggtatc cccacgcgcc ctgtagcggc gcattaagcg cggcgggtgt |
| 4081 | ggtggttacg cgcagcgtga ccgctacact tgccagcgcc ctagcgcccg ctcctttcgc |
| 4141 | tttcttccct tcctttctcg ccacgttcgc cggctttccc cgtcaagctc taaatcgggg |
| 4201 | gctcccttta gggttccgat ttagtgcttt acggcacctc gaccccaaaa aacttgatta |
| 4261 | gggtgatggt tcacgtagtg ggccatcgcc ctgatagacg gtttttcgcc ctttgacgtt |
| 4321 | ggagtccacg ttctttaata gtggactctt gttccaaact ggaacaacac tcaaccctat |
| 4381 | ctcggtctat tcttttgatt tataagggat tttgccgatt tcggcctatt ggttaaaaaa |
| 4441 | tgagctgatt taacaaaaat ttaacgcgaa ttaattctgt ggaatgtgtg tcagttaggg |
| 4501 | tgtggaaagt ccccaggctc cccagcaggc agaagtatgc aaagcatgca tctcaattag |
| 4561 | tcagcaacca ggtgtggaaa gtccccaggc tccccagcag gcagaagtat gcaaagcatg |
| 4621 | catctcaatt agtcagcaac catagtcccg cccctaactc cgcccatccc gcccctaact |
| 4681 | ccgcccagtt ccgcccattc tccgccccat ggctgactaa ttttttttat ttatgcagag |
| 4741 | gccgaggccg cctctgcctc tgagctattc cagaagtagt gaggaggctt ttttggaggc |
| 4801 | ctaggctttt gcaaaaagct cccgggagct tgtatatcca ttttcggatc tgatcaagag |
| 4861 | acaggatgag gatcgtttcg catgattgaa caagatggat tgcacgcagg ttctccggcc |
| 4921 | gcttgggtgg agaggctatt cggctatgac tgggcacaac agacaatcgg ctgctctgat |
| 4981 | gccgccgtgt tccggctgtc agcgcagggg cgcccggttc tttttgtcaa gaccgacctg |
| 5041 | tccggtgccc tgaatgaact gcaggacgag gcagcgcggc tatcgtggct ggccacgacg |
| 5101 | ggcgttcctt gcgcagctgt gctcgacgtt gtcactgaag cgggaaggga ctggctgcta |
| 5161 | ttgggcgaag tgccggggca ggatctcctg tcatctcacc ttgctcctgc cgagaaagta |
| 5221 | tccatcatgg ctgatgcaat gcggcggctg catacgcttg atccggctac ctgcccattc |
| 5281 | gaccaccaag cgaaacatcg catcgagcga gcacgtactc ggatggaagc cggtcttgtc |
| 5341 | gatcaggatg atctggacga agagcatcag gggctcgcgc cagccgaact gttcgccagg |
| 5401 | ctcaaggcgc gcatgcccga cggcgaggat ctcgtcgtga cccatggcga tgcctgcttg |
| 5461 | ccgaatatca tggtggaaaa tggccgcttt tctggattca tcgactgtgg ccggctgggt |
| 5521 | gtggcggacc gctatcagga catagcgttg gctacccgtg atattgctga agagcttggc |
| 5581 | ggcgaatggg ctgaccgctt cctcgtgctt tacggtatcg ccgctcccga ttcgcagcgc |
| 5641 | atcgccttct atcgccttct tgacgagttc ttctgagcgg gactctgggg ttcgaaatga |
| 5701 | ccgaccaagc gacgcccaac ctgccatcac gagatttcga ttccaccgcc gccttctatg |
| 5761 | aaaggttggg cttcggaatc gttttccggg acgccggctg gatgatcctc cagcgcgggg |
| 5821 | atctcatgct ggagttcttc gcccacccca acttgtttat tgcagcttat aatggttaca |
| 5881 | aataaagcaa tagcatcaca aatttcacaa ataaagcatt tttttcactg cattctagtt |
| 5941 | gtggtttgtc caaactcatc aatgtatctt atcatgtctg tataccgtcg acctctagct |
| 6001 | agagcttggc gtaatcatgg tcatagctgt ttcctgtgtg aaattgttat ccgctcacaa |
| 6061 | ttccacacaa catacgagcc ggaagcataa agtgtaaagc ctggggtgcc taatgagtga |
| 6121 | gctaactcac attaattgcg ttgcgctcac tgcccgcttt ccagtcggga aacctgtcgt |
| 6181 | gccagctgca ttaatgaatc ggccaacgcg cggggagagg cggtttgcgt attgggcgct |
| 6241 | cttccgcttc ctcgctcact gactcgctgc gctcggtcgt tcggctgcgg cgagcggtat |
| 6301 | cagctcactc aaaggcggta atacggttat ccacagaatc aggggataac gcaggaaaga |
| 6361 | acatgtgagc aaaaggccag caaaaggcca ggaaccgtaa aaaggccgcg ttgctggcgt |
| 6421 | ttttccatag gctccgcccc cctgacgagc atcacaaaaa tcgacgctca agtcagaggt |
| 6481 | ggcgaaaccc gacaggacta taaagatacc aggcgtttcc ccctggaagc tccctcgtgc |
| 6541 | gctctcctgt tccgaccctg ccgcttaccg gatacctgtc cgcctttctc ccttcgggaa |
| 6601 | gcgtggcgct ttctcatagc tcacgctgta ggtatctcag ttcggtgtag gtcgttcgct |
| 6661 | ccaagctggg ctgtgtgcac gaaccccccg ttcagcccga ccgctgcgcc ttatccggta |
| 6721 | actatcgtct tgagtccaac ccggtaagac acgacttatc gccactggca gcagccactg |
| 6781 | gtaacaggat tagcagagcg aggtatgtag gcggtgctac agagttcttg aagtggtggc |
| 6841 | ctaactacgg ctacactaga agaacagtat ttggtatctg cgctctgctg aagccagtta |
| 6901 | ccttcggaaa aagagttggt agctcttgat ccggcaaaca aaccaccgct ggtagcggtt |
| 6961 | tttttgtttg caagcagcag attacgcgca gaaaaaaagg atctcaagaa gatcctttga |
| 7021 | tcttttctac ggggtctgac gctcagtgga acgaaaactc acgttaaggg attttggtca |
| 7081 | tgagattatc aaaaaggatc ttcacctaga tccttttaaa ttaaaaatga agttttaaat |
| 7141 | caatctaaag tatatatgag taaacttggt ctgacagtta ccaatgctta atcagtgagg |
| 7201 | cacctatctc agcgatctgt ctatttcgtt catccatagt tgcctgactc cccgtcgtgt |
| 7261 | agataactac gatacgggag ggcttaccat ctggccccag tgctgcaatg ataccgcgag |
| 7321 | acccacgctc accggctcca gatttatcag caataaacca gccagccgga agggccgagc |
| 7381 | gcagaagtgg tcctgcaact ttatccgcct ccatccagtc tattaattgt tgccgggaag |
| 7441 | ctagagtaag tagttcgcca gttaatagtt tgcgcaacgt tgttgccatt gctacaggca |
| 7501 | tcgtggtgtc acgctcgtcg tttggtatgg cttcattcag ctccggttcc caacgatcaa |
| 7561 | ggcgagttac atgatccccc atgttgtgca aaaaagcggt tagctccttc ggtcctccga |
| 7621 | tcgttgtcag aagtaagttg gccgcagtgt tatcactcat ggttatggca gcactgcata |
| 7681 | attctcttac tgtcatgcca tccgtaagat gcttttctgt gactggtgag tactcaacca |
| 7741 | agtcattctg agaatagtgt atgcggcgac cgagttgctc ttgcccggcg tcaatacggg |
| 7801 | ataataccgc gccacatagc agaactttaa aagtgctcat cattggaaaa cgttcttcgg |
| 7861 | ggcgaaaact ctcaaggatc ttaccgctgt tgagatccag ttcgatgtaa cccactcgtg |
| 7921 | cacccaactg atcttcagca tcttttactt tcaccagcgt ttctgggtga gcaaaaacag |
| 7981 | gaaggcaaaa tgccgcaaaa aagggaataa gggcgacacg gaaatgttga atactcatac |
| 8041 | tcttcctttt tcaatattat tgaagcattt atcagggtta ttgtctcatg agcggataca |
| 8101 | tatttgaatg tatttagaaa aataaacaaa taggggttcc gcgcacattt ccccgaaaag |
| 8161 | tgccacctga cgtc |

The nucleic acid sequence of a vector encoding Flag-Atoh1-ER Fusion (SEQ ID NO: 16) is depicted below:

| | |
|---|---|
| 1 | gcgcatggta ccgccaccat ggactacaaa gacgatgatg ataaagatta caaagatgac |
| 61 | gatgacaagg ggtcccgcct gctgcatgca gaagagtggg ctgaggtaaa agagttgggg |
| 121 | gaccaccatc gccatcccca gccgcaccac gtcccgccgc tgacgccaca gccacctgct |
| 181 | accctgcagg cgagagacct tcccgtctac ccggcagaac tgtccctcct ggatagcacc |
| 241 | gacccacgcg cctggctgac tcccactttg cagggcctct gcacggcacg cgccgcccag |
| 301 | tatctgctgc attctcccga gctgggtgcc tccgaggccg cggcgccccg ggacgaggct |
| 361 | gacagccagg gtgagctggt aaggagaagc ggctgtggcg gcctcagcaa gagccccggg |
| 421 | cccgtcaaag tacgggaaca gctgtgcaag ctgaagggtg gggttgtagt ggacgagctt |
| 481 | ggctgcagcc gccagcgagc cccttccagc aaacaggtga atggggtaca gaagcaaagg |
| 541 | aggctggcag caaacgcaag ggaacggcgc aggatgcacg ggctgaacca cgccttcgac |
| 601 | cagctgcgca acgttatccc gtccttcaac aacgacaaga agctgtccaa atatgagacc |
| 661 | ctacagatgg cccagatcta catcaacgct ctgtcggagt tgctgcagac tcccaatgtc |
| 721 | ggagagcaac cgccgccgcc cacagcttcc tgcaaaaatg accaccatca ccttcgcacc |
| 781 | gcctcctcct atgaaggagg tgcgggcgcc tctgcggtag ctggggctca gccagccccg |
| 841 | ggagggggcc cgagacctac cccgcccggg ccttgccgga ctcgcttctc aggcccagct |
| 901 | tcctctgggg gttactcggt gcagctggac gctttgcact tcccagcctt cgaggacagg |
| 961 | gccctaacag cgatgatggc acagaaggac ctgtcgcctt cgctgcccgg gggcatcctg |
| 1021 | cagcctgtac aggaggacaa cagcaaaaca tctcccagat cccacagaag tgacggagag |
| 1081 | ttttcccccc actctcatta cagtgactct gatgaggcca gtctcgagcc atccaattta |
| 1141 | ctgaccgtac accaaaattt gcctgcatta ccggtcgatg caacgagtga tgaggttcgc |
| 1201 | aagaacctga tggacatgtt cagggatcgc caggcgtttt ctgagcatac ctggaaaatg |
| 1261 | cttctgtccg tttgccggtc gtgggcggca tggtgcaagt tgaataaccg gaaatggttt |
| 1321 | cccgcagaac ctgaagatgt tcgcgattat cttctatatc ttcaggcgcg cggtctggca |
| 1381 | gtaaaaacta tccagcaaca tttgggccag ctaaacatgc ttcatcgtcg gtccgggctg |
| 1441 | ccacgaccaa gtgacagcaa tgctgtttca ctggttatgc ggcggatccg aaaagaaaac |
| 1501 | gttgatgccg gtgaacgtgc aaaacaggct ctagcgttcg aacgcactga tttcgaccag |
| 1561 | gttcgttcac tcatggaaaa tagcgatcgc tgccaggata tacgtaatct ggcatttctg |
| 1621 | gggattgctt ataacaccct gttacgtata gccgaaattg ccaggatcag ggttaaagat |
| 1681 | atctcacgta ctgacggtgg gagaatgtta atccatattg gcagaacgaa aacgctggtt |
| 1741 | agcaccgcag gtgtagagaa ggcacttagc ctgggggtaa ctaaactggt cgagcgatgg |
| 1801 | atttccgtct ctggtgtagc tgatgatccg aataactacc tgttttgccg ggtcagaaaa |
| 1861 | aatggtgttg ccgcgccatc tgccaccagc cagctatcaa ctcgcgccct ggaagggatt |
| 1921 | tttgaagcaa ctcatcgatt gatttacggc gctaaggatg actctggtca gagatacctg |
| 1981 | gcctggtctg gacacagtgc ccgtgtcgga gccgcgcgag atatggcccg cgctggagtt |
| 2041 | tcaataccgg agatcatgca agctggtggc tggaccaatg taaatattgt catgaactat |
| 2101 | atccgtaacc tggatagtga aacaggggca atggtgcgcc tgctggaaga tggcgatctc |
| 2161 | gagccatctg ctggagacat gagagctgcc aacctttggc caagcccgct catgatcaaa |
| 2221 | cgctctaaga agaacagcct ggccttgtcc ctgacggccg accagatggt cagtgccttg |
| 2281 | ttggatgctg agccccccat actctattcc gagtatgatc ctaccagacc cttcagtgaa |
| 2341 | gcttcgatga tgggcttact gaccaacctg gcagacaggg agctggttca catgatcaac |
| 2401 | tgggcgaaga gggtgccagg ctttgtggat ttgaccctcc atgatcaggt ccaccttcta |
| 2461 | gaatgtgcct ggctagagat cctgatgatt ggtctcgtct ggcgctccat ggagcaccca |
| 2521 | gtgaagctac tgtttgctcc taacttgctc ttggacagga accagggaaa atgtgtagag |
| 2581 | ggcatggtgg agatcttcga catgctgctg gctacatcat ctcggttccg catgatgaat |
| 2641 | ctgcagggag aggagtttgt gtgcctcaaa tctattattt tgcttaattc tggagtgtac |
| 2701 | acatttctgt ccagcaccct gaagtctctg gaagagaagg accatatcca ccgagtcctg |
| 2761 | gacaagatca cagacacttt gatccacctg atggccaagg caggcctgac cctgcagcag |
| 2821 | cagcaccagc ggctggccca gctcctcctc atcctctccc acatcaggca catgagtaac |
| 2881 | aaaggcatgg agcatctgta cagcatgaag tgcaagaacg tggtgcccct ctatgacctg |
| 2941 | ctgctggagg cggcggacgc ccaccgccta catgcgccca ctagccgtgg aggggcatcc |
| 3001 | gtggaggaga cggaccaaag ccacttggcc actgcgggct ctacttcatc gcattccttg |
| 3061 | caaaagtatt acatcacggg ggaggcagag ggtttccctg ccacagcttg agcggccgca |
| 3121 | tgcgc |

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCES

The following documents are cited herein.
1. Bermingham, N. A., et al., Math1: An Essential Gene for the Generation of Inner Ear Hair Cells. Science, 1999. 284(5421): p. 1837-1841.
2. Helms, A. W., et al., Autoregulation and multiple enhancers control Math1 expression in the developing nervous system. Development, 2000. 127(6): p. 1185-96.
3. Isaka, F., et al., Ectopic expression of the bHLH gene Math1 disturbs neural development. European Journal of Neuroscience, 1999. 11(7): p. 2582-2588.
4. Jeon, S.-J., et al., Bone marrow mesenchymal stem cells are progenitors in vitro for inner ear hair cells. Molecular and Cellular Neuroscience 2007. 34(1): p. 59-68
5. Zheng, J. L. and W. Q. Gao, Overexpression of Math1 induces robust production of extra hair cells in postnatal rat inner ears. Nature Neuroscience, 2000. 3(6): p. 580-6.
6. Kawamoto, K., et al., Math1 Gene Transfer Generates New Cochlear Hair Cells in Mature Guinea Pigs In Vivo. J. Neurosci., 2003. 23(11): p. 4395-4400.
7. Danielian, P. S., et al., Modification of gene activity in mouse embryos in utero by a tamoxifen-inducible form of Cre recombinase. Curr Biol, 1998. 8(24): p. 1323-6.
8. Danielian, P. S., et al., Identification of residues in the estrogen receptor that confer differential sensitivity to estrogen and hydroxytamoxifen. Mol Endocrinol, 1993. 7(2): p. 232-40.
9. Oshima, K., et al., Differential Distribution of Stem Cells in the Auditory and Vestibular Organs of the Inner Ear. Journal of the Association for Research in Otolaryngology, 2007. 8(1): p. 18-31.
10. Parker, M. A., A. Brugeaud, and A. S. Edge, Primary culture and plasmid electroporation of the murine organ of Corti. Journal of Visualized Experiments, 2010(36).
11. Rio, C., et al., Glial fibrillary acidic protein expression and promoter activity in the inner ear of developing and adult mice. Journal of Comparitive Neurology, 2002. 442(2): p. 156-62.
12. Hume, C. R., D. L. Bratt, and E. C. Oesterle, Expression of LHX3 and SOX2 during mouse inner ear development. Gene Expr Patterns, 2007. 7(7): p. 798-807.
13. Bermingham-McDonogh, O., et al., Expression of Prox1 during mouse cochlear development. Journal of Comparitive Neurology, 2006 496(2): p. 172-86.
14. Parker, M. A., et al., TAK1 expression in the cochlea: a specific marker for adult supporting cells. J Assoc Res Otolaryngol, 2011. 12(4): p. 471-83.
15. Parker, M. A., et al., The Potential Use of Stem Cells for Cochlear Repair. Audiol Neurotol, 2004. 9: p. 72-80.
16. Bossuyt et al., Atonal homolog 1 is a tumor suppressor gene. PLoS Biol. 2009. 7(2):e39.

What is claimed is:

1. An isolated nucleic acid comprising a sequence that encodes a polypeptide comprising Atonal homolog 1 (Atoh1) or fragment thereof operably linked to an estrogen receptor (ER) or fragment thereof, wherein the Atoh1 or fragment thereof can bind nucleic acid and can activate transcription, and wherein the ER or fragment thereof can bind an ER ligand.

2. The isolated nucleic acid of claim 1, wherein the Atoh1 or fragment thereof and the ER or fragment thereof are linked by a linker.

3. The isolated nucleic acid of claim 1, wherein the ER or fragment thereof is operatively linked to the C-terminus of the Atoh1 or fragment thereof.

4. The isolated nucleic acid of claim 1, wherein the polypeptide further comprises a reporter selected from the group consisting of DsRed, GFP, RFP, BFP, CFP, and YFP.

5. The isolated nucleic acid of claim 1, wherein the reporter is linked to the Atoh1 or fragment thereof or the ER or fragment thereof by a linker.

6. The isolated nucleic acid of claim 5, wherein the reporter is operatively linked to the C-terminus of the ER or fragment thereof.

7. The isolated nucleic acid of claim 1, wherein the ER or fragment thereof has been modified to limits endogenous 17b-estradiol binding at physiological concentrations.

8. The isolated nucleic acid of claim 1, wherein the ER ligand is selected from the group consisting of 4-hydroxy Tamoxifen, Tamoxifen, and estrogen.

9. The isolated nucleic acid of claim 1, wherein the polypeptide localizes to the nucleus when contacted with an ER ligand.

10. A vector comprising the nucleic acid of any one of claim 1.

11. The vector of claim 10, wherein the vector is an expression vector suitable for expression in a mammalian cell.

12. The vector of claim 11, further comprising an enhancer or promoter of a gene selected from the group consisting of Glial fibrillary acidic protein (GFAP), SRY (sex determining region Y)-box 2 (Sox2), Prospero homeobox protein 1 (prox1), and Transforming Growth Factor β-activated Kinase 1 (TAK1).

13. A virus comprising the vector of claim 10.

14. The virus of claim 13, wherein the virus is selected from the group consisting of cytomegaloviris, lentivirus, adenovirus, retrovirus, adeno-associated virus, herpesvirus, vaccinia virus, or polyoma virus.

15. A host cell comprising the vector of claim 10.

16. The host cell of claim 15, wherein the cell is in vitro, in vivo, or ex vivo.

17. The host cell of claim 15, wherein the cell is a mammalian cell.

18. The host cell of claim 17, wherein the cell is a human cell.

19. The host cell of claim 15, wherein the cell is derived from a tumor or immortalized cell line.

20. The host cell of claim 15, wherein the cell is a hair cell or cochlear cell.

21. A xenograft comprising the cell of claim 15.

22. A method for treating or preventing hearing loss in an individual, comprising administering to an individual in need thereof a pharmacologically effective dose of a pharmaceutical composition comprising a nucleic acid comprising a sequence that encodes a isolated polypeptide comprising Atonal homolog 1 (Atoh1) or fragment thereof operably linked to an estrogen receptor (ER) or fragment thereof, wherein the Atoh1 or fragment thereof can bind nucleic acid and can activate transcription, and wherein the ER or fragment thereof can bind an ER ligand.

23. The method of claim 22, wherein the hearing loss is sensorineural hearing loss.

24. A method for treating or preventing neoplasia in an individual, comprising administering to an individual in need thereof a pharmacologically effective dose of a pharmaceutical composition comprising a nucleic acid comprising a sequence that encodes a polypeptide comprising Atonal homolog 1 (Atoh1) or fragment thereof operably linked to an estrogen receptor (ER) or fragment thereof, wherein the Atoh1 or fragment thereof can bind nucleic acid and can activate transcription, and wherein the ER or fragment thereof can bind an ER ligand.

25. The method of claim 24, wherein the neoplasia is selected from the group consisting of intestinal cancer, colorectal cancer, skin cancer, brain cancers such as gliomas and medulloblasomas and neuroendocrine cancers.

26. The method of claim 22, wherein the Atoh1 or fragment thereof and the ER or fragment thereof are linked by a linker.

27. The method of claim 22, wherein the ER or fragment thereof is operatively linked to the C-terminus of the Atoh1 or fragment thereof.

28. The method of claim 22, wherein the ER or fragment thereof has been modified to limits endogenous 17b-estradiol binding at physiological concentrations.

29. The method of claim 22, wherein the ER ligand is selected from the group consisting of 4-hydroxy Tamoxifen, Tamoxifen, and estrogen.

30. The method of claim 22, wherein the polypeptide localizes to the nucleus when contacted with an ER ligand.

31. The method of claim 22, further comprising a reporter selected from the group consisting of DsRed, GFP, RFP, BFP, CFP, and YFP.

32. The method of claim 22, wherein the reporter is linked to the Atoh1 or fragment thereof or the ER or fragment thereof by a polypeptide linker.

33. The method of claim 32, wherein the reporter is operatively linked to the C-terminus of the ER or fragment thereof.

34. The method of claim 22, wherein the polypeptide is expressed from a vector that is administered to the subject, or wherein the polypeptide is electroporated directly into a cell of said individual.

35. The method of claim 34, wherein the vector is an expression vector suitable for expression in a mammalian cell.

36. The method of claim 35, wherein the mammalian cell is a human cell.

37. The method of claim 35, wherein the cell is a hair cell or cochlear cell.

38. The method of claim 34, wherein the vector is in a virus that is administered to the subject.

39. The method of claim 38, wherein the virus is selected from the group consisting of cytomegalovirus, lentivirus, adenovirus, retrovirus, adeno-associated virus, herpesvirus, vaccinia virus, or polyoma virus.

40. The method of claim 35, wherein the polypeptide is expressed in a host cell that is administered to the subject.

41. The method of claim 40, wherein the host cell is in a xenograft that is administered to the subject.
